(19) United States
(12) Patent Application Publication
(10) Pub. No.: US 2026/0311880 A1
(43) Pub. Date: Oct. 8, 2026

(54) GENOMIC PROFILING OF CELLS TO MONITOR MINIMUM RESIDUAL DISEASE

(71) Applicants: DANA-FARBER CANCER INSTITUTE, INC., Boston, MA (US); THE GENERAL HOSPITAL CORPORATION, Boston, MA (US)

(72) Inventors: Irene Ghobrial, Boston, MA (US); Jean-Baptiste Alberge, Vanves (FR); Rosa Toenges, Cambridge, MA (US); Gad Getz, Belmont, MA (US); Tomoki IEMURA, Brookline, MA (US)

(21) Appl. No.: 19/679,357

(22) Filed: May 15, 2026

Related U.S. Application Data
(63) Continuation of application No. PCT/US2025/053913, filed on Nov. 4, 2025.
(60) Provisional application No. 63/716,165, filed on Nov. 4, 2024.

(51) Int. Cl. G16H 50/20; G16B 20/20

(52) U.S. Cl. CPC G16H 50/20; G16B 20/20

(57) ABSTRACT

Disclosed are methods of monitoring minimum residual disease using genomic profiling. In some methods, a subject has or has had multiple myeloma or a precursor thereof. The methods include amplifying a sample using primary template-directed whole genome amplification (PTWGA) and enable monitoring of liquid samples, such as blood samples.

### CROSS-REFERENCE TO RELATED APPLICATIONS

This application claims the benefit of U.S. Provisional Application Ser. No. 63/716,165, filed on Nov. 4, 2024, the disclosure of which is incorporated by reference herein in its entirety.

### STATEMENT REGARDING FEDERALLY SPONSORED RESEARCH OR DEVELOPMENT

This invention was made with government support under R35 CA263817 awarded by the National Institutes of Health. The government has certain rights in the invention.

### BACKGROUND

Multiple myeloma (MM) is a hematologic malignancy characterized by the abnormal proliferation of clonal plasma cells in the bone marrow. Patients with a relapsed/refractory MM may receive targeted immunotherapies such as anti-GPRC5D and anti-BCMA chimeric antigen receptor T cells and bispecific antibodies. While it is established that some mutations can cause baseline or acquired resistance to these types of targeted treatment, these mutations are not typically identified in the clinic. Detecting these mutations may require a bone marrow biopsy, which is often not performed in advanced disease stages. Therefore, many treatment decisions are made without knowledge of whether the patient has a mutation that would likely cause resistance.

The detection of minimal residual disease (MRD) provides valuable insight into a patient's depth of response to therapy, particularly when any residual disease markers are below the level of detection of standard monitoring tools (e.g., M-spike, Free Light Chain, Bone Marrow Plasma cells). MRD is also used as a secondary endpoint for drug approval in MM and can be used as the primary endpoint for accelerated approval.

### SUMMARY

Provided herein, in one aspect, are methods of whole-genome sequencing (WGS) of minimal numbers of cells from liquid samples (e.g., peripheral blood samples), for example, in relapsed/refractory MM (RRMM) subject, to inform clinical decisions without the need for bone marrow biopsies, including in cases where cells of interest are ultra rare (~1 in a million events). Methods of the present disclosure are particularly suited for minimal residual disease monitoring.

There are provided herein methods of monitoring minimum residual disease (MRD) in a subject diagnosed and/or treated with a condition comprising MM or a precursor thereof. The methods include separating one or more cells of interest from a liquid sample using fluorescence-activated cell sorting (FACS) to provide a cell sample, wherein the cell sample comprises the one or more cells. In this initial step if no cells of interest are separated, the subject is considered MRD negative. If cells of interest are separated, the subject may or may not be MRD positive. Subsequent to this separating step, the methods include performing primary template-directed whole genome amplification on the cell sample to provide an amplified genome and identifying one or more gene abnormalities associated with MM or a precursor thereof to determine if the subject is MRD positive. These abnormalities (e.g., mutations) mean the subject may be MRD positive depending on the abnormality. This information can be used to modify treatment or determine efficacy (i.e., effectivity) of a treatment (e.g., drug).

### BRIEF DESCRIPTION OF THE DRAWINGS

Certain illustrations, charts, or flow charts are provided to allow for a better understanding this disclosure. It is to be noted, however, that the drawings illustrate only selected embodiments and are therefore not to be considered limiting of scope. Additional and equally effective embodiments and applications of the present exist.

FIG. 1 shows a schematic representation of an example of a method consistent with some methods of the present disclosure.

FIG. 2 shows an example of a gating strategy used to isolate CD138+/CD38+/CD56+/BCMA+/CD19−/CD45−/SLAMF7+ cells from a sample including circulating tumor cells from peripheral blood of a patient with RRMM who did not respond to immunotherapy.

FIG. 3 shows an example of a gating strategy used to isolate CD138+/CD38+/BCMA+/CD56−/CD19−/CD45−/SLAMF7+ cells from a sample including circulating tumor cells from peripheral blood of a patient with untreated smoldering multiple myeloma (SMM).

FIG. 4 shows an example of a gating strategy used to isolate CD138dim/CD38+/CD56−/CD19−/CD45+ cells from a sample including circulating tumor cells from peripheral blood of a patient with RRMM in complete remission following immunotherapy.

FIG. 5 shows a Phylogenetic tree reconstruction from somatic mutations of circulating tumor cells before (Ultra RT5 4 5 CTC 591) and after (others) targeted immunotherapy, showing 2/7 cells post treatment are false positive CTCs (no mutation shared with the pre-treatment), while 5/7 share the bulk of mutations with the tumor and are real CTCs captured at clinically assessed complete response time point.

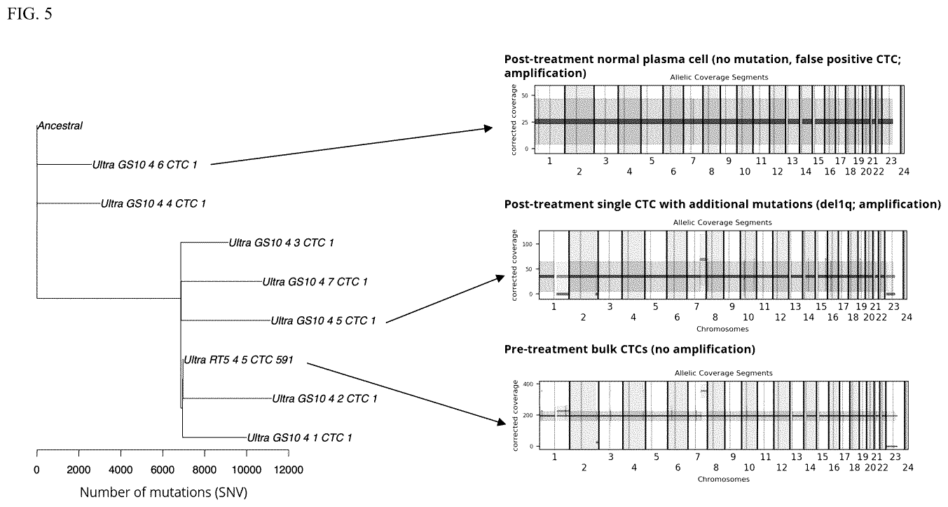

FIG. 6 shows a schematic representation of sequencing analysis methods applied to analyze ultra rare circulating tumor cells in some methods of the present disclosure.

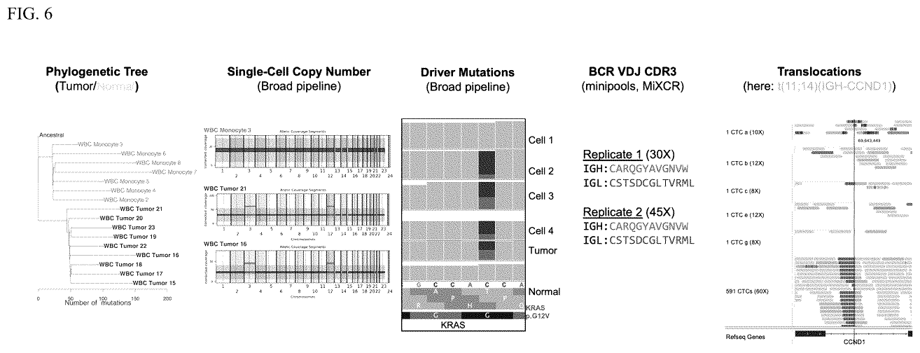

FIG. 7 shows plots identifying and mapping suspected subclonal mutations and palindromic mutations. (A) Violin plots showing variant allele frequency (vaf) on X and Y chromosomes from male, single-cell samples. (B) Mutations as a function of palindrome length before and after the location of the mutation. The horizontal axis shows the length of the longest palindrome in the germline and the vertical axis shows the length of the longest palindrome in the tumor. Both subclonal and clonal mutations were mapped.

FIG. 8 shows comparisons of palindromic and subclonal mutations. (A) Plot showing that the majority of palindromic mutations were found to have a vaf of less than 0.75. (B) Histograms showing that the majority of identified palindromes were associated with subclonal mutations.

FIG. 9 shows a plot comparing mutations identified in a sample prepared using the BioSkryb sequencing library preparation kit or synthetic mutations as a function of palindrome length before and after the location of the mutation. Random mutations in the genome were rarely found within palindromes.

FIG. 10 shows a histogram showing the length of random and BioSkryb-introduced palindromes.

FIG. 11 shows a histogram comparing the number of mutations identified when reading windows of 20 bp, 40 bp, 60 bp, and 80 bp were used to filter out palindrome-associated mutations.

FIG. 12 shows a comparison of the number of palindromes filtered out of tumor samples prepared using the BioSkryb library preparation kit and a New England Biolabs kit.

FIG. 13 shows a schematic diagram of the patient population analyzed in Example 3.

FIG. 14 shows a schematic diagram of the treatment type and timing of the patient population analyzed in Example 3.

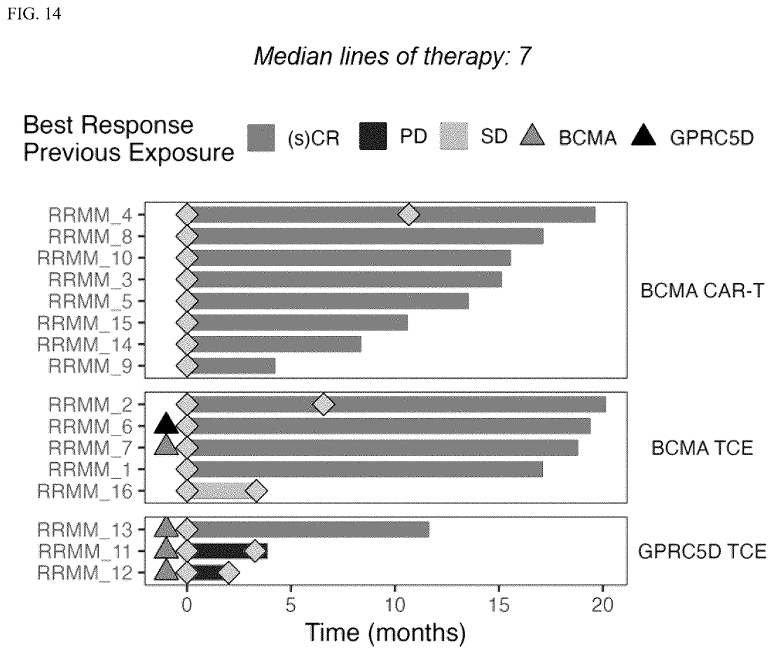

FIG. 15 shows a graph of the false positive CTC call rate as a function of the number of cells sequenced. The median number of CTCs sequenced per patient was 50 (range: 5-10,263); median tumor purity was 99% (range: 0-100%); and there was a 91% concordance between CTC WGS and clinical FISH reports observed.

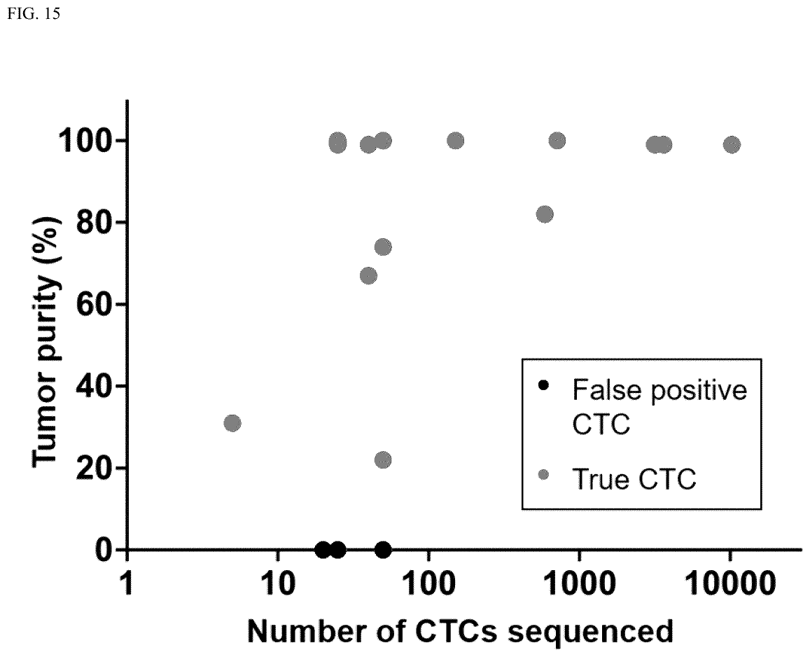

FIG. 16 shows a schematic diagram of the genomic landscape observed in CTCs from RRMM patients. Point mutations in known myeloma drivers, including KRAS, NRAS, TP53, and TRAF3 were observed. Additional mutations associated with MM were observed, including deletion 1p, amplification 1q, and deletion 13. No mutations in BCMA were observed, including in four patients who had received BCMA-targeted immunotherapy. A single copy deletion of GPCR5D was observed in one patient who had been identified as refractory to GPRC5D T cell engager therapy, suggesting reliable correlation between clinical findings and genomic data collected using CTC WGS.

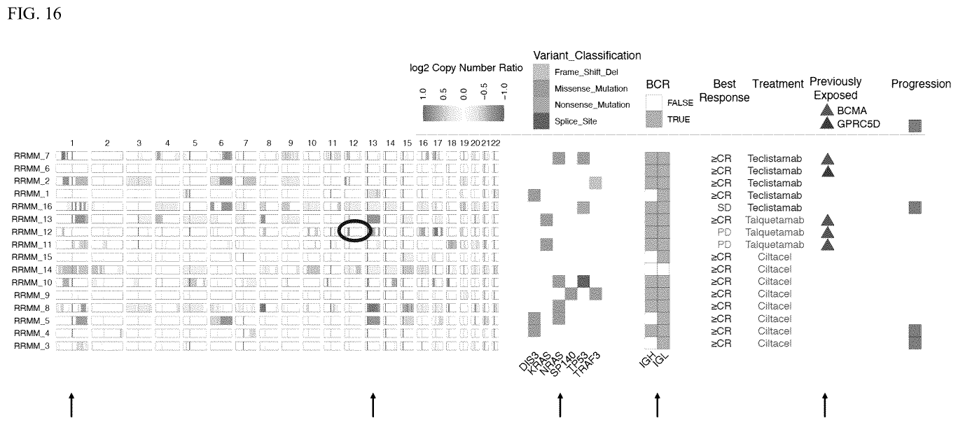

FIG. 17 shows a schematic representation of CTC WGS monitoring of patients through tumor evolution. By performing CTC WGS before, during, and/or after treatment, changes in mutations can be actively monitored and resistant subclones can be identified before the patient shows clinical symptoms. Three patients with clinically stable or progressive disease showed an overalls table tumor genome. In one patient with a complete response to treatment and no signs of clinical progression, a loss-of-function TRAF3 mutation was initially identified in 68% of cells at baseline, but this mutation was not detected after treatment, suggesting that the patient had a complete response to treatment (RMM_2). Interestingly, one patient achieved a stringent complete response, but cells having the DIS3R780K mutation were identified in a baseline sample and a sample taken after treatment initiation.

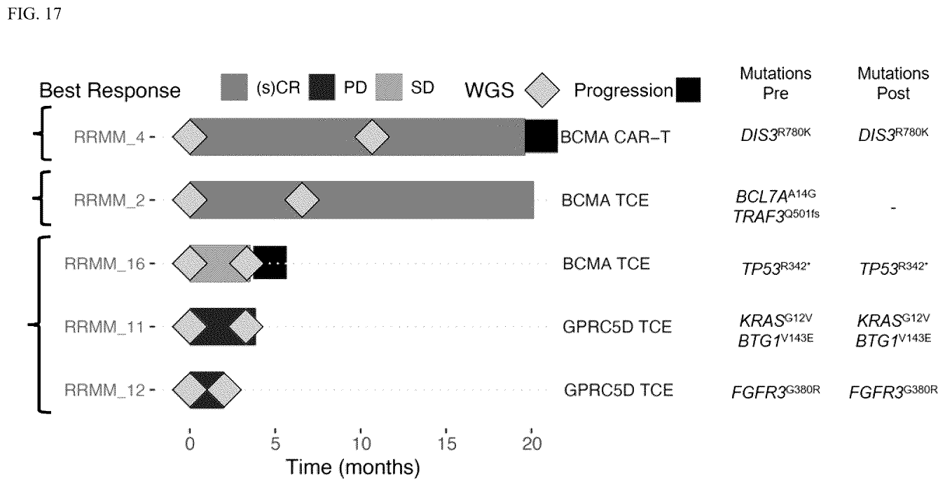

FIG. 18 shows IgA lambda MM progression and genomic profile of a patient who initially showed complete remission following treatment but relapsed 20 months after infusion. Using CTC WGS and ancestral reconstruction, it was identified that the relapse likely originated from mature residual tumor cells that survived treatment, rather than from earlier stem-like clones.

FIG. 19 shows M spike progression and genomic profile of a patient with IgA lambda transplant-ineligible MM. Cytogenetic analysis by WGS revealed gain(1q), hyperdiploidy, and deletion 17p. The patient was refractory to 8 prior lines of therapy and had not been previously exposed to an anti-BCMA agent. After beginning treatment, the patient initially achieved a stable disease response but progressed after only one month of therapy. Prior to therapy initiation, a minor clone carrying a R342* nonsense mutation inTP53 was identified. This clone increased significantly in tumor fraction during therapy, rising from 16% to 45% of the tumor population within one month. This indicates clonal expansion under selective pressure. This also suggests that clonal selection during targeted immunotherapy is not limited to mutations in therapeutic targets, such as BCMA or GPRC5D.

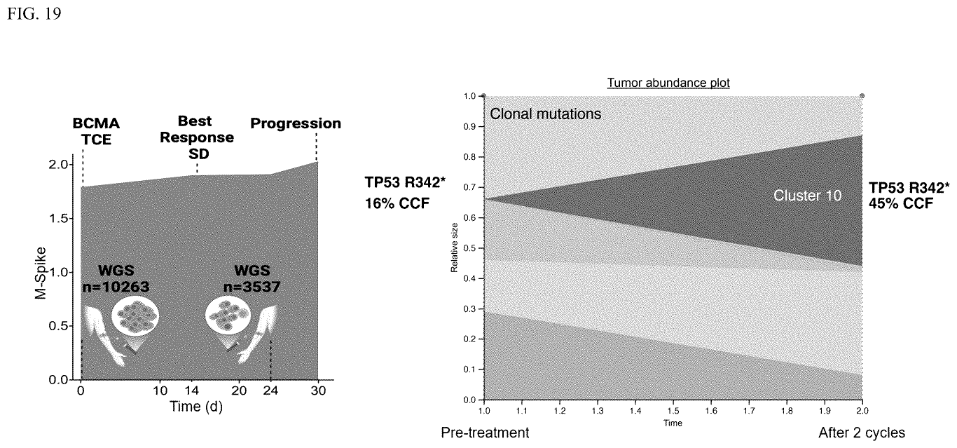

### DETAILED DESCRIPTION

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 10 percent up or down (higher or lower).

### Multiple Myeloma and Precursors Thereof

Multiple myeloma (MM) is a plasma cell dyscrasia. Plasma cell dyscrasias are cancers of the plasma cells. They are produced as a result of malignant proliferation of a monoclonal population of plasma cells that may or may not secrete detectable levels of a monoclonal immunoglobulin or paraprotein commonly referred to as M protein. Further non-limiting examples of plasma cell dyscrasias include monoclonal gammopathy of undermined significance (MGUS), smoldering multiple myeloma (SMM), and symptomatic multiple myeloma. MGUS, smoldering MM, and symptomatic MM represent a spectrum of the same disease.

Monoclonal Gammopathy of Undermined Significance (MGUS) MGUS is characterized by a serum monoclonal protein (<30 g/L), <10% plasma cells in the bone marrow, and absence of end-organ damage. Asymptomatic MGUS stage consistently precedes multiple myeloma (MM). MGUS is present in 3% of persons >50 years and in 5%>70 years of age. The risk of progression to MM or a related disorder is 1% per year. Patients with risk factors consisting of an abnormal serum free light chain ratio, non-immunoglobulin G (IgG) MGUS, and an elevated serum M protein (≥15 g/1) have a risk of progression at 20 years of 58%, compared with 37% among patients with two risk factors, 21% for those with one risk factor, and 5% for individuals with no risk factors. The cumulative probability of progression to active MM or amyloidosis is 51% at 5 years, 66% at 10 years and 73% at 15 years; the median time to progression was 4.8 years.

Smoldering Multiple Myeloma (SMM) also known as asymptomatic MM is characterized by having a serum immunoglobulin (Ig) G or IgA monoclonal protein of 30 g/L or higher and/or 10% or more plasma cells in the bone marrow but no evidence of end-organ damage. Not intending to be bound by theory, there are 2 different types of SMM: evolving smoldering MM and non-evolving Smoldering MM. Evolving SMM is characterized by a progressive increase in M protein and a shorter median time to progression (TTP) to active multiple myeloma of 1.3 years. Non-evolving SMM has a more stable M protein that can then change abruptly at the time of progression to active multiple myeloma, with a median TTP of 3.9 years.

Symptomatic or Active Multiple myeloma (MM) is a form of cancer that affects a type of white blood cell called the plasma cell. Multiple myeloma appears in the bone marrow, which is the soft tissue inside the bones that makes stem cells. In multiple myeloma, plasma cells, which mature from stem cells and typically produce antibodies to fight germs and other harmful substances, become abnormal. These abnormal cells are called myeloma cells. In 2021, an estimated 34,920 cases of multiple myeloma were diagnosed in the United States and over 12,410 patient deaths associated with multiple myeloma were reported. As the most common type of plasma cell cancer, effective treatment requires an accurate diagnosis and precise treatment.

In embodiments, symptomatic or active MM is characterized by any level of monoclonal protein and the presence of end-organ damage that consists of the CRAB criteria (hypercalcemia, renal insufficiency, anemia, or bone lesions). In some instances, multiple myeloma diagnosis is made using the detection of a biomarker for a myeloma defining event, as described, for example, in Rajkumar, S., et al., The Lancet Oncology, 15: E538-548 (2014), doi: 10.1016/S1470-2045 (14) 70442-5, the disclosure of which is incorporated herein by reference in its entirety for all purposes. MM is a plasma cell malignancy that characteristically involves extensive infiltration of bone marrow (BM), with the formation of plasmacytomas, as clusters of malignant plasma cells inside or outside of the BM milieu. Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia.

The criteria for the diagnosis of MM, SMM, and MGUS are detailed in Table 1 below. Distinction among these disease states informs treatment decisions and prognostic recommendations.

TABLE 1
Conventional criteria for the diagnosis of MM, SMM, and MGUS

| Disorder | Disease definition |
|---|---|
| MGUS | Serum monoclonal protein level <3 g/dL, bone marrow plasma cells 10%, and |
| | absence of end-organ damage, such as lytic bone lesions, anemia, hypercalcemia, |
| | or renal failure, that can be attributed to a plasma cell proliferative disorder. |
| SMM | Serum monoclonal protein (IgG or IgA) level ≥3 g/dL and/or bone marrow plasma |
| | cells .10%, absence of end-organ damage, such as lytic bone lesions, anemia, |
| | hypercalcemia, or renal failure that can be attributed to a plasma cell |
| | proliferative disorder. |
| | Alternatively or additionally, SMM may be defined by the presence or absence of |
| | biomarkers (e.g., Myeloma Defining Event biomarkers), by bone-marrow plasma |
| | cell infiltration ≥60%, by serum-free light chain ratio ≥100, and/or by >1 focal |
| | lesion in the skeleton on magnetic resonance imaging analysis (see, Rajkumar S V, |
| | et al. International Myeloma Working Group updated criteria for the diagnosis of |
| | multiple myeloma. Lancet Oncol 15, e538-548 (2014)). In some |
| | instances, SMM may be associated with organ damage. |
| MM | Bone marrow plasma cells ≥10%, presence of serum and/or urinary monoclonal |
| | protein (except in patients with true non-secretory multiple myeloma), plus |
| | evidence of lytic bone lesions, anemia, hypercalcemia, or renal failure that can |
| | be attributed to the underlying plasma cell proliferative disorder. |

Purification and/or Counting of Cells

Rather than analyzing a solid bone marrow biopsy sample directly, the methods provided can be used to measure MRD by characterizing the genomes of MM cells isolated from a liquid sample (e.g., the peripheral blood) of a subject. Such characterization is facilitated by the isolation of cells (e.g., circulating tumor cells (CTCs), tumor cells in a bone marrow aspirate) from a sample (e.g., a liquid biopsy, such as a peripheral blood sample). In bone marrow aspiration, a needle is used to withdraw a sample of the fluid portion of the bone marrow. In bone marrow biopsy (i.e., core biopsy), a needle is used to withdraw a sample of the solid portion. The present methods are suitable for use of the bone marrow aspirate.

In embodiments, genomic DNA from the cells is isolated and sequenced. In various embodiments, the MM cells are purified using an immunophenotype-based enrichment technique, such as Fluorescence-activated cell sorting (FACS) or CellSearch™.

In embodiments, the methods of the disclosure involve enumeration of cells. Such enumeration can be used for characterizing disease state (e.g., multiple myeloma (MM), smoldering multiple myeloma (SMM), monoclonal gammopathy of undermined significance (MGUS)).

The detection of minimal residual disease (MRD) provides valuable insight into a patient's depth of response to therapy, particularly when any residual disease markers are below the level of detection of standard monitoring tools (e.g., M-spike, Free Light Chain, Bone Marrow Plasma cells). MRD is used as a secondary endpoint for drug approval in MM and can be used as the primary endpoint for accelerator approval. As an endpoint, MRD can be tested either by multiparametric flow cytometry (MFC) or next-generation sequencing (NGS). MRD is tested on cells taken from the liquid samples (e.g., peripheral blood cells or tumor cells from bone marrow aspirate), and assays are typically sensitive to a level of 10-5 or better (<105) in patients who have achieved a complete response (CR) as defined by the International Myeloma Working Group guidelines (Table 4). MRD assessment by NGS typically requires a baseline sample (sometimes referred to as the “calibration” sample) that determines the hypervariable sequences of the immunoglobulin genes that are monitored over time, although a baseline is not necessary for use in the present methods.

### Samples

Traditional methods of whole genome sequencing require a relatively high-quality sample. For example, some methods of whole genome sequencing require a freshly isolated biopsy of a solid tissue or tumor. The present methods may be compatible with frozen samples or with samples including a relatively low number of cells. Additionally or alternatively, the present methods may be compatible with targeted analysis of rare cells from a population of many cells. As is described in Example 1, methods such as magnetic cell isolation and fluorescence-activated cell sorting (FACS) may be paired with primary template-directed whole genome amplification (PTWGA) and sequencing (e.g., NGS) methods to yield surprisingly high-quality sequencing results from a low-quality or low-frequency sample.

In methods of the present disclosure, liquid samples are used. In certain methods, a liquid sample is a liquid biopsy. For example, a sample may include peripheral blood. In certain methods, a liquid sample is a bone marrow aspirate.

Typically, the sample is taken from a subject (e.g., a patient) with multiple myeloma or a precursor thereof. This may include smoldering multiple myeloma (SMM), newly diagnosed multiple myeloma (NDMM), relapsed/refractory multiple myeloma (RRMM), or a combination thereof. Typically, the sample is compared to a matched control sample, such as a germline sample from the same subject. Some methods include a germline cell from a matched subject. Some method may alternatively or additionally include a sample from a healthy subject, such as a subject who does not have a hematological malignancy or a subject in remission from a hematological malignancy.

Advantageously, the present methods are typically compatible with banked samples. The sample may be frozen, such as a sample stored at −80° C. The sample may be of any suitable age, such as over one year old. Typically, the sample has been frozen such that the cells are viable after thawing. Viably frozen cells are often stored in a solution including fetal bovine serum (FBS) with 10% dimethyl sulfoxide (DMSO).

The cell of interest may be, for example, a CTC or a tumor cell from a bone marrow aspirate.

In addition, methods of the present disclosure are suitable for use with a relatively low number of cells. The sample including the cells of interest prior to cell processing (e.g., FACS) is sometimes referred to as the “subject sample” or “liquid sample” or “liquid biological sample.” The subject sample may include a biopsy, such as a liquid biopsy. The subject sample may include 10⁰ cells or fewer, 90 cells or fewer, 80 cells or fewer, 70 cells or fewer, 60 cells or fewer, 50 cells or fewer, 40 cells or fewer, 30 cells or fewer, 25 cells or fewer, or 20 cells or fewer.

In various aspects, the disclosure provides methods that involve isolation or enrichment of a small number of purified cells (e.g., about or at least about 2 cells, 3 cells, 4 cells, 5 cells, 6 cells, 7 cells, 8 cells, 9 cells, 10 cells, 20 cells, 30 cells, 40 cells, 50 cells, 60 cells, 70 cells, 80 cells, 90 cells, 100 cells, 200 cells, 300 cells, 400 cells, 500 cells, 600 cells, 700 cells, 800 cells, 900 cells, 1000 cells, 2000 cells, 3000 cells, 4000 cells, 5000 cells, 6000 cells, 7000 cells, 8000 cells, 9000 cells, 1000 cells, 2000 cells, 3000 cells, 4000 cells, 5000 cells, 6000 cells, 7000 cells, 8000 cells, 9000 cells, 1000 cells, 10000 cells, 20000 cells, 30000 cells, 40000 cells, 50000 cells, 60000 cells, 70000 cells, 80000 cells, 90000 or 100000 cells).

In embodiments, a collection of sorted cells (e.g., a “minipool”) contains about or at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 1000, 2000, 3000, 4000, 5000, 6000, 7000, 8000, 9000, 1000, 10000, 20000, 30000, 40000, 50000, 60000, 70000, 80000, 90000 or 100000 cells (e.g., cells and/or plasma cells). In some instances, the collection of sorted cells contains no more than about 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 1000, 2000, 3000, 4000, 5000, 6000, 7000, 8000, 9000, 1000, 10000, 20000, 30000, 40000, 50000, 60000, 70000, 80000, 90000 or 100000 cells (e.g., circulating tumor cells and/or plasma cells). In embodiments, the collection of sorted cells contains a fraction of cells of interest equal to about or at least about 5%, 10%, 15%, 20%, 25%, 30%, 35%, 40%, 45%, 50%, 55%, 60%, 65%, 70%, 75%, 80%, 85%, 90%, 95%, 99%, or 100%. In some instances, the collections of sorted cells include only plasma cells (e.g., CTC). In embodiments, the collection of sorted cells contains no more than about 10, 100, 1000, or 10000 leukocytes and/or other non-plasma cells. In various embodiments, the cells are intact cells.

The subject sample may include a relatively low proportion of cells of interest. For example, a sample of peripheral blood may include many cells, such as immune cells and red blood cells, but may include relatively few cells of interest. In methods wherein the circulating tumor cells or tumor cells in bone marrow aspirate are of primary interest, it may be challenging to isolate relatively few cells from a sample including many other cells. In some methods, at most 1%, at most 0.1%, at most 0.01%, at most 0.001%, or at most 0.0001% of the total number of cells in the subject sample may be cells of interest. In some methods, fewer than one in 1,000, one in 10,000, one in 100,000, one in 500,000, one in 1,000,000, one in 10,000,000, or one in 50,000,000 of the total cells in the subject sample may be cells of interest.

The sample including the cells of interest after cell processing but before nucleic acid processing (e.g., whole genome amplification) is sometimes referred to as the “cell sample.” Typically, the cell sample will include fewer cells than the subject sample. The cell sample may include 50 cells or fewer, 40 cells or fewer, 30 cells or fewer, 25 cells or fewer, 20 cells or fewer, 15 cells or fewer, 10 cells or fewer, 8 cells or fewer, 6 cells or fewer, 5 cells or fewer, 4 cells or fewer, 3 cells or fewer, 2 cells, or 1 cell.

The cell sample is typically processed to provide a nucleic acid for further analysis, such as sequencing. The methods of the present disclosure are advantageous in that they are suitable for processing a relatively small amount of nucleic acid. The cell sample may include a total of at most 1000 picograms (pg), 800 pg, 500 pg, 300 pg, 200 pg, 100 pg, 50 pg, 40 pg, 30 pg, 20 pg, 10 pg, or 6 pg of nucleic acid. Without wishing to be bound by theory, it is believed that each cell from a sample may provide approximately 6 pg of DNA.

The nucleic acid analyzed using methods of the present disclosure is typically DNA, particularly genomic DNA (gDNA). gDNA presents unique challenges for analysis from small samples, and sequencing and analysis of a whole genome typically requires a large, high-quality sample. The methods of the present disclosure advantageously allow for high-quality analysis of gDNA from relatively low-quality samples.

### Methods of Identifying and Detecting

In one aspect, there are provided methods of identifying one or more gene abnormalities in a sample. These methods typically include providing a liquid sample, such as a liquid biopsy, wherein the sample includes one or more cells of interest. The one or more cells of interest may be separated using any of the methods of cell sorting described herein, preferably magnetic cell sorting and FACS. Separating the cells provides a cell sample, which includes the one or more cells of interest. The cell sample is then prepared for sequencing using any of the methods of nucleic acid preparation and processing described herein. gDNA is provided from the cell sample and the gDNA is amplified using PTWGA. The nucleic acid, such as the amplified gDNA is then sequenced to provide sequencing data. The sequencing data is then typically analyzed to identify one or more gene abnormalities associated with MM or a precursor thereof. The one or more gene abnormalities identified may be any described herein as being associated with MM or a precursor thereof, and preferably include a translocation, a copy number variation (CNV), and/or a single nucleotide variation (SNV).

In one aspect, the present disclosure describes methods of monitoring MRD in a subject (e.g., patient) having been diagnosed with and/or treated for MM or a precursor thereof. These methods typically include providing a sample from the subject, such as a liquid biopsy, wherein the sample includes one or more cells of interest. Optionally, the liquid sample is concentrated in plasma cells using magnetic beads.

The one or more cells of interest may be separated using any of the methods of cell sorting described herein, preferably magnetic cell sorting and FACS. Separating the cells provides a cell sample, which includes the one or more cells of interest. The cell sample is then prepared for sequencing using any of the methods of nucleic acid preparation and processing described herein. Preferably, gDNA is provided from the cell sample and the gDNA is amplified using PTWGA. The nucleic acid, such as the amplified gDNA is then sequenced to provide sequencing data. The sequencing data is then typically analyzed to identify one or more gene abnormalities associated with MM or a precursor thereof. The one or more gene abnormalities identified may be any described herein as being associated with MM or a precursor thereof, and preferably include a translocation, an SNV, and/or a CNV.

### Cell Selection

Methods of the present disclosure typically include separating one or more cells of interest from a sample. Any known method of cell selection may be used, but magnetic cell separation and FACS are preferred.

Magnetic cell separation may include positive magnetic selection, where one or more cells of interest are bound by and subsequently released from a magnetic affinity column. Such an approach may be used to isolate, for example, CD138+ cells.

Cell sorting (e.g. FACS) may be used to identify and separate a population of cells based on expression or lack of expression of multiple cell surface markers. Some methods use more than one fluorescently labeled antibody to label and subsequently sort cells. Labeling with a marker is typically quantified as being negative (−) or positive (+), with positive marker labeling being subdivided into dim, low, medium, and high or bright labeling. In this way, a cell that is considered to be, for example, CD138+ may also be CD138dim, but a sample that is CD138dim may not be considered CD138high. Typically, a population is annotated as dim/low when its fluorescence is found at lower levels than in a positive control. For example, when a cell population having a fluorescence intensity of 103 may be annotated as dim/low when compared to a positive control that shows fluorescence intensity of 10⁴-10⁵.

Markers of interest to methods of the present disclosure include CD138, CD38, CD56, CD319, CD19, CD45, and CD269. A method may use one or more of these markers to select a population of cells of interest. Some methods include selecting cells that are CD138+. Some methods include selecting cells that are CD138dim. A cell may be CD138dim, rather than CD138+, due to antigen shedding upon freezing and thawing or entering apoptosis. A cell may be CD38dim, rather than CD38+, due to prior lines of treatments with an anti-CD38 monoclonal antibody (e.g. daratumumab or isatuximab). Some methods include selecting cells that are CD38+. Some methods include selecting cells that are CD45+. Some methods include selecting cells that are CD45−. CD45 status may vary by patient. Some methods include selecting cells that are CD19−.

Some methods include selecting cells that are CD56+. Some methods include selecting cells that are CD56−. Whether cells expressing or lacking CD56 are selected may be determined based on prior clinical data from the same sample source (e.g., subject).

Some methods include selecting cells that are CD269+.

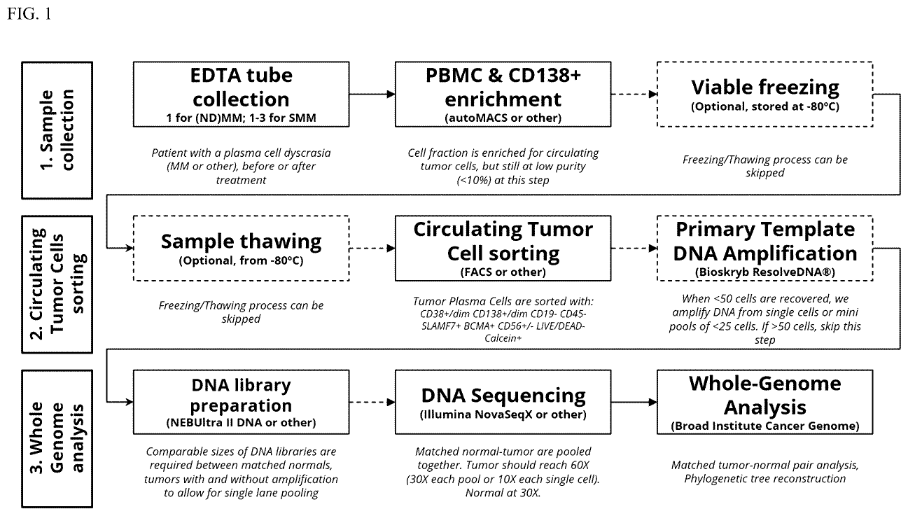

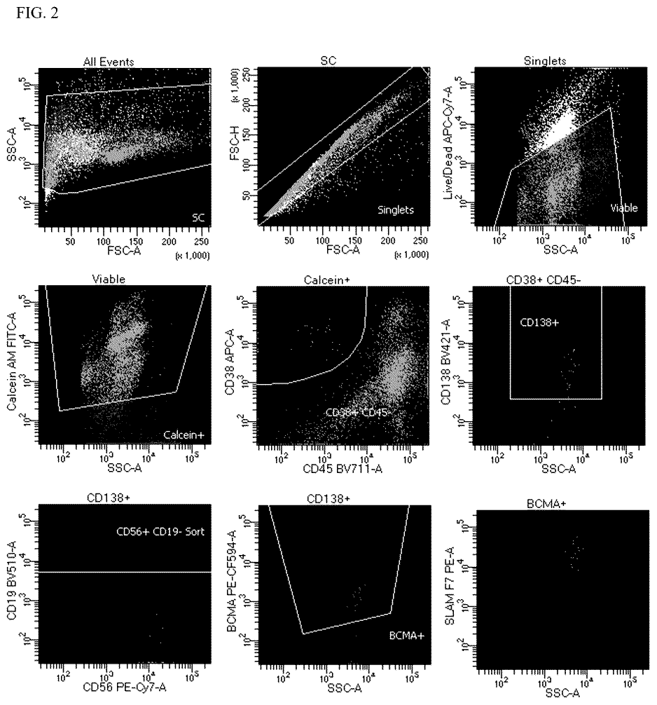

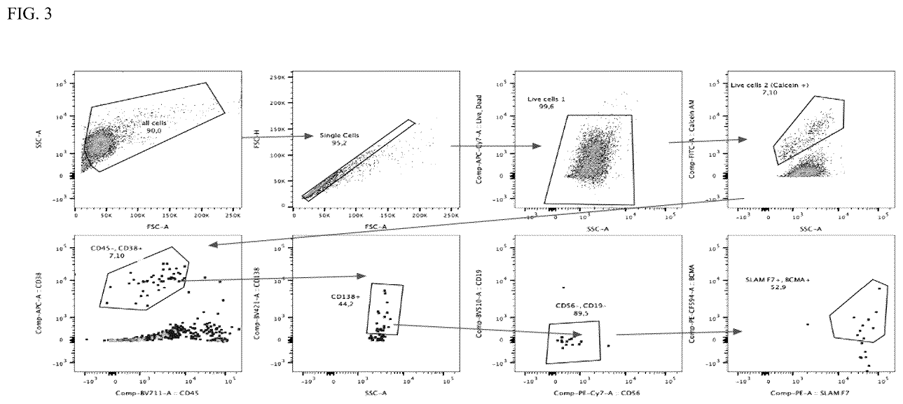

A method may include separating one or more cells of interest that are CD138+/CD38+/CD56+/BCMA+/CD19−/CD45−. An example of a FACS strategy to separate these type of cells is shown in FIG. 1. In another example, a method may include separating one or more cells of interest that are CD138+/CD38+/BCMA+/CD56−/CD19−/CD45−. An example of a FACS strategy to separate these type of cells is shown in FIG. 2. In another example, a method may include separating one or more cells of interest that are CD138dim/CD38+/BMCAdim/CD56−/CD19−/CD45+. An example of a FACS strategy to separate these type of cells is shown in FIG. 3. Some other methods may include separating one or more cells of interest that are CD38+/CD138+/CD269+/CD19−/CD45−/CD319+ and may be CD56+ or CD56−.

A sample may additionally be labeled to identify live cells. Methods may include labeling a sample with one or more live/dead stains, such as Near-IR (APC Cy7) LIVE/DEAD stain, Calcein (FITC) LIVE/DEAD stain, or both. Without wishing to be bound by theory, use of more than one live/dead stain may improve sample quality by allowing more stringent removal of dead cells.

### Primary Template-Directed Whole Genome Amplification

As is described in Example 1, methods of analysis of the present disclosure advantageous allow for accurate identification of gene abnormalities from a relatively small and/or low-quality sample. In particular, methods of analysis described herein allow reliable identification of gene abnormalities when samples have been processed using a particular type of whole genome amplification (WGA). Historically, WGA has introduced amplification bias, artifacts, allelic distortions, and non-uniformity in genome coverage. However, the methods of the present disclosure utilize a specific sub-type of WGA called PTWGA such that these errors and artifacts are not introduced or may be removed during analysis of the sequencing results. Thus, these methods of analysis are uniquely suited for analysis of samples amplified using this sub-type WGA.

More specifically, the methods of WGA used herein include primary template-directed amplification, such as primary template-directed whole genome amplification (PTWGA). Methods of PTWGA are described, for example, in WO2019148119A1. PTWGA uses exonuclease-resistant terminators to create relatively short, double-stranded amplification products that are poor templates for subsequent cycles of amplification. This limits the propagation of amplification bias, allelic skewing, and the propagation of replication errors.

Although PTWGA is particularly well suited for amplification of a single cell, in the present method, 1 cell or more may be used. Some methods of the present disclosure use PTWGA to amplify a minipool of cells, such as a minipool of 25 cells or fewer. PTWGA was tested on pooled samples of 1, 2, 5, 10, 20, 25, 36, 50, or 10⁰ cells. The amplified sample from each pool was then sequenced. For pools of greater than 25 cells, known heterozygous germline variants were not detected. Detection of such variants is required for ensuring cells are of good quality (i.e., ensuring that there is adequate coverage of the genome, allelic distortion is minimized, and false positive mutations are not introduced). Preferably, samples of at most 25 cells are used.

Methods typically include sequencing the nucleic acid. When the nucleic acid is amplified, it is sequenced after PTWGA. Prior to sequencing, a library may be prepared using methods known to the art.

In embodiments the provided methods involve sequencing genomic DNA obtained from a collection of sorted cells prepared according to the methods provided herein. In embodiments, the method for sequencing the genomic DNA does not involve any whole-genome amplification step.

In embodiments of the provided methods, next-generation sequencing (NGS) of genomic DNA from cells isolated or enriched from a liquid biopsy sample allows for capture of the genetic abnormalities of MM, similar to that detected in standard BM biopsy sample analysis alone. The methods enable quantitative disease monitoring for subjects in the clinic at regular intervals. In various instances, the methods involve enriching and selecting cells from a large background of mononuclear cells, and subsequently extracting nucleic acids from this minute cell fraction to allow for subsequent molecular characterization of the cells.

Any suitable method for isolation of DNA from the cells may be used in the present methods (e.g., proteinase K-based purification methods). Various kits are commercially available for the purification of polynucleotides from a sample and are suitable for use in the methods (e.g., an Arcturus PicoPure DNA Extraction Kit, Thermo Fisher Scientific). In an embodiment, the genomic DNA is purified using a proteinase K digestion-based technique (e.g., Arcturus PicoPure DNA Extraction Kit, Thermo Fisher Scientific)

In embodiments, the extracted DNA is used to prepare a sequencing library. Methods for preparing libraries of polynucleotides for sequencing are known to one of skill in the art. Library preparation can include the addition of nucleotide bar codes to the library polynucleotides according to methods known in the art. Libraries can be prepared using commercially available kits.

The extracted DNA may be sequenced using any high-throughput platform. Methods of sequencing oligonucleotides and nucleic acids are well known in the art.

The sequencing of a polynucleotide and/or sequencing library can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing. In embodiments, the polynucleotide is sequenced using HiSeq2500 or Novaseq6000.

In embodiments, the sequencing is to a coverage of about or at least about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×, where a sequencing coverage of 0.01 indicates that a DNA sample has been sequenced such that the amount of DNA sequenced is equivalent in size to about 1% of the corresponding genome from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×.

In embodiments, the methods of the disclosure further involve analyzing sequence data obtained through the sequencing of a polynucleotide and/or sequencing library. The analysis can involve the detection of clinically relevant and/or prognostic events, such as driver mutations, single nucleotide variation, and/or chromosomal rearrangements (e.g., structural variation or copy number variation) associated with a multiple myeloma. Non-limiting examples of clinically relevant and/or prognostic events that may be detected include aneuploidy (e.g., hyperploidies, such as trisomies, or tetrasomies; and monoploidies), translocations (e.g., t(4;14), t(6;14), t(8;14), t(11;14), t(14;16), t(14;20), t(2;8), and t(8;22)), chromosomal arm gains or deletions (e.g., chromosome 1q gain, chromosome 1p deletion, chromosome 13q deletion, chromosome 16q deletion, and chromosome 17p deletion), and/or driver mutations (e.g., non-silent mutations to KRAS or NRAS, and/or mutations to DIS3, FAM46C, BRAF, and/or TP53). The event detected can be predictive of risk and MM progression. In embodiments, a driver mutation is selected from a G12, G13, Q61, K117, or A146 alteration to KRAS or NRAS. In some instances, translocations are detected using density-based graph clustering of sequencing reads supporting oncogenic structural rearrangements. In embodiments, structural rearrangements are detected using sequencing read pairs or single sequencing reads from both chromosomes of a translocation (e.g., from both chromosome 11 and chromosome 14). In embodiments, a driver mutation is selected from any non-silent mutation (e.g., a G12, G13, Q61, K117, or A146 alteration) to KRAS or NRAS. In some instances, a driver mutation is selected from any non-silent mutation to any one or more of the following genes: KRAS, NRAS, DIS3, BRAF, FAM46C, TP53, MYC, MAX, IGLL5, TRAF3, DUSP2, TCL1A, TRAF2, CYLD, LTB, HIST1H1E, BCL7A, SP140, NFKBIA, EGR1, PABPC1, PRKD2, TBC1D29, IRF4, RB1, TGDS, PTPN11, FUBP1, RPL5, FGFR3, SAMHD1, ACTG1, HIST1H1B, NFKB2, KMT2B, KLHL6, RASA2, PIM1, PRDM1, DTX, SETD2, BHLHE41, RPL10, BTG1, RPS3A, CCND1, RPRD1B, HIST1H1D, ZNF292, RFTN1, CDKN1B, LCE1D, XBP1, IRF1, POTI, HIST1H2BK, ABCF1, ZFP36L1, TET2, ARID2, KDM6A, EP 300, ARID1A, NCOR1, HUWE1, CDKN2C, SF3B1, ATM, NF1, CREBBP, DNMT3A, MAFB, MAF, KDM5C, UBR5, PIK3CA, IDH1, MCL1, BIRC2, MLL1, MLL2, MAML2, MAN2C1, IDH2, KMT2C, and ATRX.

The sequence data obtained according to the methods allows for the detection and quantification of genetic abnormalities in genomic DNA of cells of interest from subjects with hematological malignancies (e.g., monoclonal gammopathies).

### Analysis of Sequencing Results

As is described in Example 1, methods of analysis of the present disclosure advantageous allow for accurate identification of gene abnormalities from a relatively small and/or low-quality sample. In particular, methods of analysis described herein allow reliable identification of gene abnormalities when samples have been processed using whole genome amplification (WGA). Historically, WGA has introduced amplification bias, artifacts, allelic distortions, and non-uniformity in genome coverage. However, the methods of the present disclosure utilize PTWGA such that these errors and artifacts are not introduced or may be removed during analysis of the sequencing results. Thus, these methods of analysis are uniquely suited for analysis of samples amplified using PTWGA.

Methods of the present disclosure typically include analyzing sequencing results. One or more analysis methods may be used, such as those described below. When a sample includes one or more genetic abnormalities, they are typically identified by the analysis method(s). The method may then further include identifying that the one or more genetic abnormalities are associated with MM or a precursor thereof.

Mutations were detected with MuTect (Cibulskis K, Lawrence M S, Carter S L, Sivachenko A, Jaffe D, Sougnez C, Gabriel S, Meyerson M, Lander E S, Getz G. Sensitive detection of somatic point mutations in impure and heterogeneous cancer samples. Nature biotechnology. 2013; 31 (3): 213-9) for single nucleotide variants (SNV), and with Strelka2 (Kim S, Scheffler K, Halpern AL, Bekritsky MA, Noh E, Källberg M, Chen X, Kim Y, Beyter D, Krusche P. Strelka2: fast and accurate calling of germline and somatic variants. Nature methods. 2018; 15 (8): 591-4) for small insertions and deletions (indels). Candidate mutants were filtered through a Panel of Normals consisting of PBMCs processed with the same library preparation protocol, for orientation bias inducing artifact (oxoG, FFPE, Costello M, Pugh TJ, Fennell TJ, Stewart C, Lichtenstein L, Meldrim J C, Fostel J L, Friedrich D C, Perrin D, Dionne D. Discovery and characterization of artifactual mutations in deep coverage targeted capture sequencing data due to oxidative DNA damage during sample preparation. Nucleic acids research. 2013; 41 (6): e67-e), for mapping artifacts with the BLAT realignment, and for enzymatic shearing cruciform variant artifacts. deTiN was used to estimate tumor-in-normal (TiN) contamination rate and rescue candidate mutations accordingly under 10% TiN (Taylor-Weiner A, Stewart C, Giordano T, Miller M, Rosenberg M, Macbeth A, Lennon N, Rheinbay E, Landau D-A, Wu CJ. DeTiN: overcoming tumor-in-normal contamination. Nature methods. 2018; 15 (7): 531-4). Fingerprinting was used to confirm matching tumor-normal pairing (Javed N, Farjoun Y, Fennell TJ, Epstein CB, Bernstein BE, Shoresh N. Detecting sample swaps in diverse NGS data types using linkage disequilibrium. Nature Communications. 2020; 11 (1): 3697). ConEst (Cibulskis K, McKenna A, Fennell T, Banks E, DePristo M, Getz G. ContEst: estimating cross-contamination of human samples in next-generation sequencing data. Bioinformatics. 2011; 27 (18): 2601-2) was used to detect exogenous contamination. Candidate mutations were also matched against the bovine genome with BLAT to ensure samples were not contaminated from other species (Wienand K, Chapuy B, Stewart C, Dunford A J, Wu D, Kim J, Kamburov A, Wood T R, Cader F Z, Ducar M D. Genomic analyses of flow-sorted Hodgkin Reed-Sternberg cells reveal complementary mechanisms of immune evasion. Blood advances. 2019; 3 (23): 4065-80). Finally, candidate mutations were annotated with GATK Funcotator (v1.7).

Allelic copy number ratios were calculated with the HapASeg method which clusters allelic imbalance obtained from tumor VAF of heterozygous SNPs found in the matched normal and copy-ratio segments from total depth of coverage ratio between tumor and matched normal. ABSOLUTE (Carter S L, Cibulskis K, Helman E, McKenna A, Shen H, Zack T, Laird P W, Onofrio R C, Winckler W, Weir B A. Absolute quantification of somatic DNA alterations in human cancer. Nature biotechnology. 2012; 30 (5): 413-21) was used to estimate purity, ploidy, and cancer cell fraction of mutations and copy number ratios and all solutions were manually reviewed. When several samples were available for the same participant, subclonal structures were deciphered with the PhylogicNDT suite of tools (Leshchiner I, Livitz D, Gainor J F, Rosebrock D, Spiro O, Martinez A, Mroz E, Lin J J, Stewart C, Kim J, Elagina L, Bozic I, Mino-Kenudson M, Rooney M, Ou S-HI, Wu C J, Rocco J W, Engelman J A, Shaw A T, Getz G. Comprehensive analysis of tumour initiation, spatial and temporal progression under multiple lines of treatment. bioRxiv. 2019:508127. doi: 10.1101/508127, Leshchiner I, Mroz EA, Cha J, Rosebrock D, Spiro O, Bonilla-Velez J, Faquin WC, Lefranc-Torres A, Lin DT, Michaud WA, Getz G, Rocco JW. Inferring early genetic progression in cancers with unobtainable premalignant disease. Nat Cancer. 2023; 4 (4): 550-63. Epub 20230420. doi: 10.1038/s43018-023-00533-y. PubMed PMID: 37081260; PMCID: PMC10132986). When several cells were sequenced from the same samples, the Sequoia method (github.com/TimCoorens/Sequoia) was used to build a phylogenetic tree. CDR3 sequence of the tumor clone was estimated with the MiXCR method after subsetting only sequencing reads mapping to the immunoglobulin loci (Bolotin, et al. Nature Methods 2015).

Structural variants (SVs) were detected with Manta, SvABA, and dRanger/BreakPointer (Chen X, Schulz-Trieglaff O, Shaw R, Barnes B, Schlesinger F, Källberg M, Cox AJ, Kruglyak S, Saunders CT. Manta: rapid detection of structural variants and indels for germline and cancer sequencing applications. Bioinformatics. 2016; 32 (8): 1220-2. doi: 10.1093/bioinformatics/btv710, Wala JA, Bandopadhayay P, Greenwald NF, O'Rourke R, Sharpe T, Stewart C, Schumacher S, Li Y, Weischenfeldt J, Yao X. SvABA: genome-wide detection of structural variants and indels by local assembly. Genome research. 2018; 28 (4): 581-91, Drier Y, Lawrence MS, Carter SL, Stewart C, Gabriel SB, Lander ES, Meyerson M, Beroukhim R, Getz G. Somatic rearrangements across cancer reveal classes of samples with distinct patterns of DNA breakage and rearrangement-induced hypermutability. Genome research. 2013; 23 (2): 228-35). Consensus lists of candidate SVs were then established as previously described with the exception of the 10% VAF filter which was set to 0% to increase sensitivity to detect immunoglobulin rearrangements (Morton L M, Karyadi D M, Stewart C, Bogdanova TI, Dawson ET, Steinberg MK, Dai J, Hartley SW, Schonfeld SJ, Sampson JN. Radiation-related genomic profile of papillary thyroid carcinoma after the Chernobyl accident. Science. 2021; 372 (6543): eabg2538). In particular, these were filtered based on genomic locations found in Table 2 and Table 3 with variance of the read start position on both loci >0 and mapping quality of reads at the fusion partner >60. Finally, all candidate SVs from the deep WGS cohorts were manually reviewed with IGV (Robinson JT, Thorvaldsdóttir H, Winckler W, Guttman M, Lander ES, Getz G, Mesirov JP. Integrative genomics viewer. Nature Biotechnology. 2011; 29 (1): 24-6. doi: 10.1038/nbt.1754).

TABLE 2
hg38 coordinates (immunoglobulin regions)

| Chromosome | Start | End | Region |
|---|---|---|---|
| chr2 | 88736483 | 90241133 | IGK |
| chr14 | 105333663 | 106879844 | IGH |
| chr22 | 21995603 | 23077814 | IGL |

TABLE 3
hg38 coordinates (partners regions)

| Chromosome | Start | End | Region |
|---|---|---|---|
| chr4 | 1798273 | 1998273 | MMSET |
| chr6 | 7699767 | 8199767 | TXNDC5 |
| chr6 | 41632262 | 42332262 | CCND3 |
| chr8 | 126987755 | 128587754 | MYC |
| chr8 | 142918584 | 143925832 | MAFA |
| chr11 | 68532532 | 70153894 | CCND1 |
| chr12 | 3890834 | 4390834 | CCND2 |
| chr16 | 78096103 | 79966103 | MAF |
| chr17 | 45072632 | 45422634 | MAP3K14 |
| chr20 | 39621358 | 40971360 | MAFB |

As is described in Example 2, mutations may be introduced to the sample via a sample library preparation method, potentially resulting in false positive mutations. One potential source of false positive mutations is the introduction of palindromic regions during sample preparation. In particular, enzymatic library preparation methods are known to introduce false palindromes, or “pseudo-palindromes” by mutating nucleotides in quasi-palindromic sequences to be fully complementary, thereby forming a palindrome. To identify and remove false positive mutations, a method may include identifying one or more palindromic regions, identifying one or more mutations in or nearby the one or more palindromic regions, and removing the one or more mutations from analysis. A method may additionally or alternatively include identifying one or more palindromic regions in a sample that are not in a reference human genome sequence.

### Hardware and Software

The present disclosure also relates to a computer system involved in carrying out the methods of the disclosure relating to both computations and sequencing. The methods described herein, analyses can be performed on general-purpose or specially programmed hardware or software. One can then record the results (e.g., characterization of a CTC) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In aspects, software used to analyze the data can include code that applies an algorithm to the analysis of the results. The software can also use input data (e.g., sequence) to characterize CTCs.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine-readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high-capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Methods of Diagnosing and Determining Risk/Response

In another aspect, a method may be used to determine whether a subject has, or is at risk of developing, MM or a precursor thereof. These methods typically include providing a sample from the subject, such as a liquid biopsy, wherein the sample includes one or more cells of interest. The one or more cells of interest may be separated using any of the methods of cell sorting described herein, preferably magnetic cell sorting and FACS. Separating the cells provides a cell sample, which includes the one or more cells of interest. The cell sample is then prepared for sequencing using any of the methods of nucleic acid preparation and processing described herein. Preferably, gDNA is provided from the cell sample and the gDNA is amplified using PTWGA. The nucleic acid, such as the amplified gDNA is then sequenced to provide sequencing data. The sequencing data is then typically analyzed to identify one or more gene abnormalities associated with MM or a precursor thereof. The one or more gene abnormalities identified may be any described herein as being associated with MM or a precursor thereof, and preferably include a translocation, an SNV, and/or a CNV.

In another aspect, a method may be used to identify MM or a precursor thereof in a subject. These methods typically include providing a sample from the subject, such as a liquid biopsy, wherein the sample includes one or more cells of interest. The one or more cells of interest may be separated using any of the methods of cell sorting described herein, preferably magnetic cell sorting and FACS. Separating the cells provides a cell sample, which includes the one or more cells of interest. The cell sample is then prepared for sequencing using any of the methods of nucleic acid preparation and processing described herein. Preferably, gDNA is provided from the cell sample and the gDNA is amplified using PTWGA. The nucleic acid, such as the amplified gDNA is then sequenced to provide sequencing data. The sequencing data is then typically analyzed to identify one or more gene abnormalities associated with MM or a precursor thereof. The one or more gene abnormalities identified may be any described herein as being associated with MM or a precursor thereof, and preferably include a translocation, an SNV, and/or a CNV.

### Predicting Subject Response

The methods of the present disclosure may be used to predict whether a subject will respond to a particular treatment. These methods typically include providing a sample from the subject, such as a liquid biopsy, wherein the sample includes one or more cells of interest. The one or more cells of interest may be separated using any of the methods of cell sorting described herein, preferably magnetic cell sorting and FACS. Separating the cells provides a cell sample, which includes the one or more cells of interest. The cell sample is then prepared for sequencing using any of the methods of nucleic acid preparation and processing described herein. Preferably, gDNA is provided from the cell sample and the gDNA is amplified using PTWGA. The nucleic acid, such as the amplified gDNA is then sequenced to provide sequencing data. The sequencing data is then typically analyzed to identify one or more gene abnormalities associated with MM or a precursor thereof. The one or more gene abnormalities identified may be any described herein as being associated with MM or a precursor thereof, and preferably include a translocation, an SNV, and/or a CNV.

To determine whether a subject is likely to respond to a particular treatment, the genetic and cytological identity of the one or more cells of interest may be analyzed. For example, a cell that does not highly express BCMA (CD269) may not respond well to an anti-BCMA targeted therapy, such as an anti-BCMA CAR-T cell. As another example, a cell that does not highly express CD38 may not respond well to an anti-CD38 targeted therapy, such as an anti-CD38 CAR-T cell. Generally, if a mutation is identified in a gene, it may indicate that the subject likely would not respond to a therapy targeting the gene product.

### Identifying Persister Cells

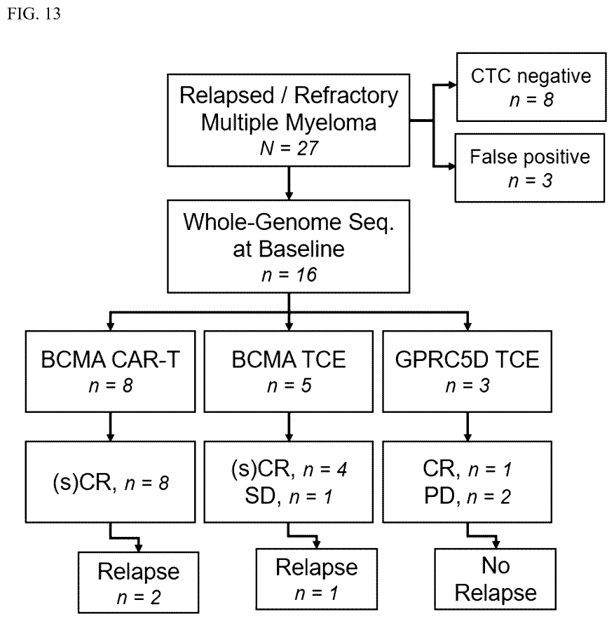

Mutations in cancer driver genes and target antigens are associated with poor prognosis in MM, but their role in minimal residual disease (MRD) and in driving progression under immunotherapy has been unclear. Example 3 describes a method of detecting the cells responsible for relapse using longitudinal single-cell whole-genome sequencing (scWGS) to isolate and characterize tumor cells—including rare MRD cells—from patients treated with quadruplet therapies (Daratumumab, Lenalidomide, Bortezomib, Dexamethasone (DRVd)), anti-BCMA-directed T-cell-redirecting therapy (Teclistamab), or anti-BCMA CAR-T cell therapy (Cilta-cel) (FIG. 13).

In another aspect, the present disclosure relates to a method to assess MRD cells using peripheral blood (PB) in conjunction with bone marrow (BM) biopsies. To select treatment methods that are more likely to be successful, it is often helpful to genetically profile the MRD cells. Example 3 of the present application describes a method to identify “persister” cells, or cells that initially appear to respond to treatment, but later expand and cause the patient to relapse. Performing WGS of cells, such as CTCs before, during, and/or after treatment, and optionally after relapse, can provide information sufficient to use ancestral reconstruction to identify the likely origin of the cells responsible for the relapse.

A method may include identifying a mutation correlated with patient clinical response. For example, where a patient is found to be clinically unresponsive to treatment with an anti-BCMA antibody, WGS may reveal that they have a mutation in the gene encoding BCMA.

### Predicting Risk of Relapse

In another aspect, a method may be used to predict the likelihood of relapse in a subject. Typically, the subject has or has had MM or a precursor thereof and has received a treatment for the hematological malignancy. The methods of the present disclosure are more sensitive relative to prior methods of analyzing samples from subjects with hematological malignancy. Thus, the methods of the present disclosure may be applied to identify one or more rare or ultrarare cells in a subject that that has been treated for MM or a precursor thereof. The subject may not exhibit clinical signs of disease following treatment, but may still harbor one or more rare or ultrarare cells that may later lead to relapse. Using traditional methods of detecting hematological malignancy, these cells would typically not be identified. However, in view of the improved sensitivity of the methods described herein, these rare or ultrarare cells may be identified using these methods. In some methods, identifying one or more rare or ultrarare cells having one or more genetic abnormalities may indicate an increased risk of relapse. Thus, some methods include predicting the likelihood of relapse in a subject.

When a method is used to identify risk of relapse, the sample may be taken immediately following treatment or a predetermined period of time following treatment. In some methods, a subject may be periodically tested to monitor risk of relapse following treatment. A sample from the subject may be tested once weekly, biweekly, monthly, bimonthly, quarterly, biannually, or annually. A method of monitoring a subject for high-risk subclones may continue for any suitable amount of time or until the risk level is determined to be lower.

### Prior Treatment

Some methods relate to predicting a subject's prognosis following treatment for MM or a precursor thereof. Thus, in some methods, a subject has previously been treated for MM or a precursor thereof. In some methods, the subject has received a bone marrow transplant.

As is described in Example 1, a patient's prior treatment may influence their cell populations, particularly if the prior treatment was targeted. In some methods, the subject has been previously treated with a CAR-T cell or a TCE, such as a CAR-T cell or a TCE targeted against one or more of CD38 or BCMA (CD269), and/or GPRC5D.

When a prior treatment is known, the method may be selected to identify particular populations of cells of interest. For example, when a subject has been treated with immunotherapy but did not respond, a method may include separating one or more cells of interest that are CD138+/CD38+/CD56+/BCMA+/CD19−/CD45−. An example of a FACS strategy to separate these type of cells is shown in FIG. 1. In another example, when a subject has an untreated hematological malignancy, a method may include separating one or more cells of interest that are CD138+/CD38+/BCMA+/CD56−/CD19−/CD45−. An example of a FACS strategy to separate these type of cells is shown in FIG. 2. In another example, when a subject has been treated for MM or a precursor thereof and is in remission, a method may include separating one or more cells of interest that are CD138dim/CD38+/BMCAdim/CD56−/CD19−/CD45+. An example of a FACS strategy to separate these type of cells is shown in FIG. 3. Some other methods may include separating one or more cells of interest that are CD38+/CD138+/CD269+/CD19−/CD45−/CD319+ and may be CD56+ or CD56−. Whether the cells of interest are CD56+ or CD56− may be determined based on previous characterization of a sample from the subject. An example of a FACS strategy to separate these type of cells is described in Example 1.

When a subject was previously treated with a CAR-T, a TCE, or both, the target of the CAR-T and/or the TCE may be differently treated during selection of the cells of interest. For example, when a subject was previously treated with a CAR-T and/or a TCE against BCMA (CD269), selecting the one or more cells of interest may include selecting cells that are CD269dim. When a subject was previously treated with a CAR-T and/or a TCE against CD38, selecting the one or more cells of interest may include selecting cells that are CD38dim.

### Continued Monitoring

Regardless of whether genetic abnormalities are identified, the method may include monitoring the subject by repeating the method periodically. For example, a new sample from a subject may be used in any of the methods described herein every three months, every six months, every nine months, or every twelve months.

Genomic characterization of the tumor cells of MM or a precursor thereof at the single cell-level post therapy, can be used to predict response to immunotherapy, monitor the emergence of high-risk subclones, and inform subsequent treatment decisions for patients with disease relapse without the need for a bone marrow biopsy.

Some methods include periodically testing samples from a subject for high-risk subclones. High-risk subclones may be identified by structural variants: t(14;16) (MAF), t(14;20) (MAFB), point mutations: KRAS (in particular at amino acid position G12, G13, Q61, A146), NRAS (G12, G13, Q61, A146), FAM46C, TP53, Del(1p), del(1p22.1), del(1p12), del(1p32), Gain(1q), gain(1q21.2), Del(4p16.3), Del(4q34.3), Del(8p), del(8p23.3), Del(8q24.21), gain(8q24.21), structural variation of MYC, and Del(17p) of Del(TP53). These markers may be detected with the analysis methods described herein structural variants: SV methods, point mutations: MuTect and Strelka+filters, copy number (del/gain) with HapASeg. A method may include obtaining a sample from a subject for use in any of the methods described herein once weekly, biweekly, monthly, bimonthly, quarterly, biannually, or annually. A method of monitoring a patient for high-risk subclones may continue for any suitable amount of time or until the risk level is determined to be lower.

Subjects being treated for MM or a precursor thereof (e.g., a monoclonal gammopathy) may be characterized using any of the methods described herein. Cells characteristic of MM or a precursor thereof typically display alterations in their genome compared to corresponding normal reference cells. Genetic alterations (e.g., mutations, chromosomal rearrangements, or aneuploidy) are correlated with multiple myeloma and related pathologies (e.g., MGUS, SMM).

A patient is evaluated for positive or negative MRD status, which is relative to the threshold of detection. Measurable levels of detection for tumor cells out of total number of cells can be 10-4, 10-5, or 10-6. In subjects that are MRD negative, no tumor cells are detectable. In subjects that are suspected of being MRD positive, cells are sorted, amplified, and sequenced to determine the presence of abnormalities associate with MRD positivity. Such MRD status is used in clinical trials to accelerate the approval of treatments (e.g., drugs). In such situations, MRD status is a surrogate for progression-free survival. That is, MRD status gives more timely input on treatment effectivity than waiting for disease progression. MRD status can also be used to select a treatment protocol.

In embodiments, the methods are used to monitor a patient. In some instances, monitoring of a subject involves characterizing cells of interest from a subject according to the methods provided herein every or at least about every 1 day, 1 week, 2 weeks, 3 weeks, 4 weeks, 5 weeks, 6 weeks, 7 weeks, 8 weeks, 9 weeks, 10 weeks, 11 weeks 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or year, optionally over a period of at least about 1 week, 2 weeks, 3 weeks, 4 weeks, 5 weeks, 6 weeks, 7 weeks, 8 weeks, 9 weeks, 10 weeks, 11 weeks 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or a year.

In these methods, a biological sample is in liquid form (e.g., a liquid biopsy) and is obtained from the subject and cells isolated from the biological sample are characterized according to the methods provided herein. The liquid biological sample can be, e.g., a body fluid such as blood or plasma, or a sample from a tumor from the subject (e.g., tumor cells from bone marrow aspirate). Typically, the liquid biological sample is a blood sample (e.g., a peripheral blood (PB) sample). An elevated number of cells of interest relative to a reference (e.g., a healthy subject) is indicative of a later stage of multiple myeloma. In embodiments, a subject with SMM has a higher level of cells of interest in a peripheral blood sample than a subject with MGUS. Also, in some embodiments, elevated cell count (e.g., count of cells of interest) in a peripheral blood sample taken from a subject relative to a reference is indicative of a higher risk stage for the multiple myeloma (e.g., a Feb. 20, 2020 risk stage). For example, in embodiments, a subject with SMM has a cell count of from about 1 to about or at least about 1, 2, 3, 4, 5, 6, 7, 8, 9, 10, 11, 12, 13, 14, 15, 16, 17, 18, 19, or 20 cells and a subject with SMM has a cell count of about or at least about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000, and a subject with MM has a cell count of about or at least about 500, 600, 700, 800, 900, 1000, 1100, 1200, 1300, 1400, 1500, or 2000. In embodiments, a low Feb. 20, 2020 risk stage is associated with a cell count of less than about 1, 2, 3, 4, 5, 6, 7, 8, 9, 10, 11, 12, 13, 14, 15, 16, 17, 18, 19, 20, 25, or 30 cells, an intermediate Feb. 20, 2020 risk is associated with a cell count of from about 5, 10, 15, 20, 25, 30, 35, 40, 45, or 50 cells to about 50, 60, 70, 80, 90, 100, 200, 300, 400, 500, 600, 700, 800, 900, 1000, or 10,000 cells, and a high Feb. 20, 2020 risk is associated with a cell count of greater than about 500, 600, 700, 800, 900, 1000, 1100, 1200, 1300, 1400, 1500, 2000, 3000, 4000, or higher. In embodiments, the cells are counted using a 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more peripheral blood sample.

Measuring the levels of the cells is also useful in determining whether a particular treatment is working for the subject. The levels of cells may be measured at any point where a health care practitioner expects the treatment the subject has been receiving to have begun to be effective in controlling the malignancy (e.g., 1 week, 2 weeks, 3 weeks, 1 month, two months, three months, four months, five months, six months, between six months and 1 year, at 1 year). The level of the cells that are found to be elevated or decreased in the MGUS, SMM, or symptomatic stage of MM are measured in the subject's biological sample obtained before and after treatment. If these levels are similar to the levels from an advanced stage of the malignancy or if the levels remain the same as the stage of the disease when the subject began treatment, the treatment is determined to have been ineffective and a new treatment is considered.

For example, consider a subject at the MGUS stage of MM who is receiving treatment for the malignancy. A biological sample is obtained before and after treatment from the subject. Tumor cell levels at the different stages of the malignancy are measured in both the pre- and post-treatment samples. If after treatment, the subject shows a cell level that resembles the SMM or active MM stage of the disease, or if the level remains unaltered compared to pre-treatment, the treatment regimen that the subject was on is deemed ineffective and a new treatment is administered to the subject. If after treatment, the subject shows a cell level that resembles a subject who does not have a multiple myeloma (e.g., MGUS, SMM, or MM) or if the level is lower than before treatment, the subject is continued on the treatment regimen that the subject was receiving.

### Methods of Treating

In another aspect, the present disclosure relates to methods of treating a subject, such as a subject. Typically, a method of treating a subject includes providing a sample from the subject for use in any of the methods of the present disclosure and later administering a treatment to the subject.

For example, a method of treating a subject may include identifying one or more gene abnormalities in a sample from the subject and administering a treatment to the subject.

Administering a treatment to the subject may include administration of any suitable treatment for MM or a precursor thereof. In some methods, the treatment is a targeted treatment, such as an antibody, a CAR-T cell, or a TCE. The CAR-T cell may be, for example, an anti-GPRC5D CAR-T cell, an anti-BCMA CAR-T cell (e.g., ciltacabtagene autoleucel, idecabtagene vicleucel), or both. The TCE may be an anti-GPRC5D TCE, an anti-BCMA TCE, or both. Examples of BCMA and CD3 bispecific antibodies include teclistamab, elranatamab, and linvoseltamab.

In some methods, the treatment includes a targeted treatment in addition to administration of lenalidomide with iberdomide or mezigdomide. In some methods, a treatment includes administering an anti-CD38 monoclonal antibody (such as daratumumab or isatuximab) with an immunomodulatory agent (such as lenalidomide) and a glucocorticoid (such as dexamethasone). When the genetic marker t(11;14) is identified, a methods may include treating the subject with Venetoclax.

In some methods, the treatment is not a targeted treatment. The treatment may be, for example, surgery, chemotherapy, or radiation therapy.

In some methods, the treatment includes a bone marrow transplant. Typically, the bone marrow transplant is an allogenic bone marrow transplant. The treatment may additionally or alternatively include a hematopoietic stem cell transplant. The hematopoietic stem cell transplant is typically an allogeneic hematopoietic stem cell transplant, although autologous hematopoietic stem cell transplants may be used where a sample is available.

In some methods, analyzing the sequencing results will reveal that the subject does not have one or more genetic abnormalities. When the subject does not have one or more genetic abnormalities, the treatment administered may be intended to encourage continued health. For example, the treatment may be a maintenance therapy. A maintenance therapy may include an immunomodulatory agent (e.g., lenalidomide), a combination of an immunomodulatory agent (e.g., lenalidomide) and a glucocorticoid (e.g., dexamethasone); Novel combinations of the above with a monoclonal antibody (e.g., daratumumab).

Methods of inhibiting and/or treating cancer and tumors (e.g., a multiple myeloma) in a subject with cancer or a predisposition for developing cancer as identified by methods of the disclosure are also contemplated. Methods described herein are useful as clinical or companion diagnostics for therapies or can be used to guide treatment decisions based on clinical response/resistance. For example, a subject having a translocation t(4;14), t(14;16), t(14;20), or del(17p) can advantageously be treated using a tandem autologous stem cell transplant (ASCT) in multiple myeloma patients, optionally instead of single ASCT (see, e.g., Kumar 2017 Nat Rev Dis Prim doi: 10.1038/nrdp.2017.46). In an embodiment, a subject having a translocation t(11;14) can advantageously be treated using Venetoclax.

In some instances, a subject with 17p or that is classified as being at high risk is treated with a tandem transplant, immunotherapy, or consolidation therapy (e.g., radiation therapy, stem cell transplant, or treatment with a chemotherapeutic agent). In some embodiments, a subject classified as being at low or intermediate risk may stop therapy without prolonged maintenance if they are in minimal residual disease (MRD).

Frontline therapy for MM includes either conventional chemotherapy or high-dose chemotherapy (HDT) supported by autologous or allogeneic stem cell transplantation (SCT), depending on patient characteristics such as performance status, age, availability of a sibling donor, comorbidities, and, in some cases, patient and physician preferences. Other treatments include: bortezomib, thalidomide, lenalidomide, dexamethasone, cyclophosphamide, melphalan, and stem cell transplant. For a patient under 70 years of age, autologous stem cell transplant is proposed after induction.

Non-limiting examples of agents suitable for use to treat a multiple myeloma include a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate. Further non-limiting examples of chemotherapeutic agents include an alkylating agent (e.g. busulfan, chlorambucil, cisplatin, cyclophosphamide (Cytoxan), dacarbazine, ifosfamide, mechlorethamine (mustargen), and melphalan), a topoisomerase inhibitor, an antimetabolite (e.g. 5-fluorouracil (5-FU), cytarabine (Ara-C), fludarabine, gemcitabine, and methotrexate), an anthracycline, an antitumor antibiotic (e.g. bleomycin, dactinomycin, daunorubicin, doxorubicin (Adriamycin), and idarubicin), an epipodophyllotoxin, nitrosureas (e.g. carmustine and lomustine), topotecan, irinotecan, doxorubicin, etoposide, mitoxantrone, bleomycin, busultan, mitomycin C, cisplatin, carboplatin, oxaliplatin and docetaxel.

In embodiments, response to therapy is measured using the methods provided herein (e.g., through molecular characterization of circulating multiple myeloma cells). In embodiments, response to therapy is measured by a reduction in M protein levels in serum and/or urine and the reduction in size or disappearance of plasmacytomas. The international uniform response criteria for MM have expanded upon the European Group for Blood and Marrow Transplantation criteria to provide a more comprehensive evaluation system (Durie B. G. et al., Leukemia, 20:1467-73 (2006)). Importantly, achievement of response has been associated with improved survival in SCT trials with high-dose therapy. Similarly, time to progression (TTP) has been shown to be an important surrogate for improved survival. Despite high response rates to frontline therapy, virtually all patients eventually relapse. Table 4 shows the international uniform response criteria for MM.

TABLE 4
International uniform response criteria for multiple myeloma (MM)

| Response | |
|---|---|
| Subcategory | Response Criteria |
| CR (complete | Negative immunofixation on the serum and urine and disappearance of |
| response) | any soft tissue plasmacytomas and ≤5% plasma cells in bone marrow |
| sCR (stringent | CR as described above, plus: |
| complete | normal free light chain (FLC) ratio and absence of clonal cells in |
| response) | bone marrow by immunohistochemistry or immunofluorescence |
| VGPR (very | Serum and urine M-protein detectable by immunofluorescence but not |
| good partial | on electrophoresis or 90% or greater reduction in serum M-protein |
| response) | plus urine M-protein level <100 mg per 24 hours |
| PR (partial | ≥50% reduction of serum M-protein and reduction in 24-h urinary |
| response) | M-protein by ≥90% or to <200 mg per 24 h |
| | If the serum and urine M-protein are unmeasurable, a ≥50% decrease |
| | in the difference between involved and uninvolved FLC levels is |
| | required in place of the M-protein criteria |
| | If serum and urine M-protein are unmeasurable, and serum free light |
| | assay is also unmeasurable, ≥50% reduction in plasma cells is |
| | required in place of M-protein, provided baseline bone marrow plasma |
| | cell percentage was ≥30% |
| | In addition to the above listed criteria, if present at baseline, |
| | a ≥50% reduction in the size of soft tissue plasmacytomas is also |
| | required. |
| SD (stable | Not meeting criteria for CR, VGPR, PR or progressive disease |
| disease) | |

In embodiments, the subject has been diagnosed with cancer or is at risk of developing a multiple myeloma.

For therapeutic use, administration of an agent can begin at the detection or surgical removal of tumors. This can be followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The disclosure provides compositions for parenteral administration which comprise a solution of a suitable agent dissolved or suspended in an acceptable carrier, preferably an aqueous carrier. A variety of aqueous carriers may be used, e.g., water, buffered water, saline, glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic (e.g., an antibody). The cancer therapeutic can be a chimeric antigen receptor (CAR) T cell. The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and US Patent Publication Nos. 20110293637, 20160008447, 20160101170, 20160331822 and 20160339090).

### Kits

The disclosure also provides kits for use in characterizing a biological sample from a subject. Kits of the instant disclosure may include one or more containers comprising an agent for enriching/isolating and/or characterization of tumor cells (e.g., circulating tumor cells or CTCs) and/or for treatment of a multiple myeloma (MM). In some embodiments, the kits further include instructions for use in accordance with the methods of this disclosure. In some embodiments, these instructions comprise a description of use of the agent to enrich/isolate and/or characterize tumor cells (e.g., CTCs) and/or use of the agent for treatment of a multiple myeloma (MM). In some embodiments, the instructions comprise a description of how to isolate polynucleotides from a sample and/or to characterize tumor cells. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present methods employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the methods. Particularly useful techniques for particular embodiments will be discussed in the sections that follow.

### Illustrative Embodiments

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:

Embodiment 1 is a method of monitoring minimum residual disease (MRD) in a subject diagnosed and/or treated for a condition including MM or a precursor thereof, the method including:

separating one or more cells of interest from a liquid sample using fluorescence-activated cell sorting (FACS) to provide a cell sample, wherein the cell sample includes the one or more cells;
performing primary template-directed whole genome amplification on the cell sample to provide an amplified genome; and
identifying one or more gene abnormalities associated with MM or a precursor thereof to determine if the subject is MRD positive.

Embodiment 2 is the method of embodiment 1, wherein the liquid sample includes a liquid sample of concentrated plasma cells.

Embodiment 3 is the method of embodiment 2, wherein the plasma cells are concentrated in the liquid sample using magnetic beads.

Embodiment 4 is the method of any preceding embodiment, wherein the cell sample includes 50 cells or fewer.

Embodiment 5 is the method of any preceding embodiment, wherein the cell sample includes one or more tumor cells.

Embodiment 6 is the method of embodiment 5, wherein the cell sample includes fewer than one cell of interest per 10,000 cells.

Embodiment 7 is the method of any preceding embodiment, wherein separating the one or more cells of interest includes labeling the one or more cells with a fluorescently labeled antibody, wherein the antibody includes affinity to CD138, CD38, CD56, CD319, CD19, CD45, CD269, or a combination thereof.

Embodiment 8 is the method of any preceding embodiment, wherein separating the one or more cells of interest from the liquid sample includes labeling the one or more cells with one or more cell viability stains.

Embodiment 9 is the method of any preceding embodiment, wherein separating the one or more cells of interest are CD45− and CD19−.

Embodiment 10 is the method of any preceding embodiment, wherein separating the one or more cells of interest are CD269+, CD319+, CD138+ or CD138dim, and CD38+ or CD38dim.

Embodiment 11 is the method of any preceding embodiment, wherein separating the one or more cells of interest from the liquid sample includes labeling the one or more cells with a live/dead stain.

Embodiment 12 is the method of any preceding embodiment, wherein the cell sample includes 300 picograms of DNA or less.

Embodiment 13 is the method of any preceding embodiment, wherein the one or more gene abnormalities are selected from the group consisting of a translocation, a copy number variation (CNV), and a single nucleotide variation (SNV).

Embodiment 14 is the method of any preceding embodiment, wherein the liquid sample includes a blood sample or a bone marrow aspirate.

Embodiment 15 is the method of any preceding embodiment, wherein identifying one or more gene abnormalities associated with MM or a precursor thereof includes:
sequencing the amplified genome; and
analyzing the sequencing results to identify the one or more gene abnormalities associated with MM or a precursor thereof.

Embodiment 16 is the method of any preceding embodiment, wherein the subject has received a bone marrow transplant.

Embodiment 17 is the method of embodiment 15, wherein sequencing the amplified genome includes using NGS.

Embodiment 18 is the method of any preceding embodiment, wherein the cell sample includes at most 1% of the cells in the liquid sample.

Embodiment 19 is the method of any preceding embodiment, further determining an effectiveness of a treatment being administered to the subject.

Embodiment 20 is the method of any preceding embodiment, further including administering a treatment to the subject.

Embodiment 21 is the method of any preceding embodiment, further including identifying one or more palindromic regions in the amplified genome, identifying one or more mutations within the one or more palindromic regions, and removing the one or more mutations from analysis.

Embodiment 22 is the method of embodiment 21, wherein the one or more palindromic regions identified are not present in a reference human genome sequence.

Embodiment 23 is the method of any preceding embodiment, wherein the subject has been previously treated for a condition including MM or a precursor thereof, been determined to be disease free, and subsequently relapsed.

### EXAMPLES

Examples are provided below to facilitate a more complete understanding of the disclosure. The following examples illustrate the exemplary modes of making and practicing what is disclosed. However, the scope of the disclosure is not limited to specific embodiments disclosed in these Examples, which are for purposes of illustration only, since alternative methods can be utilized to obtain similar results.

### Example 1—Whole Genome Sequencing (WGS) on Peripheral Blood Samples

Samples from patients with smoldering multiple myeloma (SMM), newly diagnosed multiple myeloma (NDMM), or relapsed/refractory multiple myeloma (RRMM) were collected at Dana-Farber Cancer Institute from the Precursor Crowd (PCROWD; IRB #14-174) or the Plasma Cell Dyscrasias study (IRB #07-150). Clinical characteristics of each patient were assessed at baseline, and responses to therapy were determined according to the IMWG Uniform Response Criteria for MM. All patients provided informed written consent for the research use of their peripheral blood (PB) and bone marrow (BM) samples. Research studies were carried out in accordance with the Declaration of Helsinki. PB and BM samples were drawn into EDTA tubes (8-10 mL, 1 tube for NDMM and RRMM, 1 to 3 tubes for SMM). Mononuclear cells were first enriched from PB and BM with the PBMC program of the autoMACS (SepMate, STEMCELL Technologies). Subsequently, plasma cells (PC) were enriched using CD138-coated magnetic beads and the PC program of the autoMACS. The cell counts and viability of MACS-sorted samples was then evaluated using trypan blue exclusion on an automated cell counter (Invitrogen Countess 3, Thermo Fisher Scientific). Samples were then viably frozen in fetal bovine serum (FBS) with 10% DMSO and stored at −80° C.

Samples from 16 patients with multiple myeloma were prepared for WGS. The samples were taken from patients with RRMS after a median of 6 lines of therapy (range: 3 to 12) and before receiving an anti-BCMA CAR T (n=8), an anti-BCMA TCE (n=5), or an anti GPRC5D TCE (n=3). Samples were stored at −80° C. and had been stored frozen for a variable amount of time.

PB and BM samples were then thawed (1 min, 37° C. water bath), washed with a solution of phosphate-buffered saline (PBS), 3% FBS, and centrifuged at 330 relative centrifugal force (rcf) for 5 minutes. Cell pellets were then resuspended in 100 μL of the sorting buffer (PBS, 3% FBS). Each sample was thawed and prepared for flow cytometry. CD138+ cells were enriched using autoMACS™ magnetic bead selection (Miltenyi Biotec). After magnetic bead selection, cells were labeled for fluorescence activated cell sorting (FACS) using the antibodies and stains in Table 5 below. All antibodies were from Biolegend and the live/dead stains were from Life Technologies.

TABLE 5
Antibodies and stains used for FACS

| Target | Conjugate | Clone |
|---|---|---|
| CD138 | BV-241 | MI15 |
| CD38 | APC | HB-7 |
| CD56 | PE-Cy7 | 5.1H11 |
| CD319 | PE | 132.1 |
| (SLAMF7) | | |
| CD269 | PE/Dazzle-594 | 19F2 |
| (BCMA) | | |
| CD19 | BV-510 | HIB19 |
| CD45 | FITC | HI30 |
| Live/Dead 1 | NA | NA |
| (Near-IR) | | |
| Live/Dead 2 | NA | NA |
| (Calcein) | | |

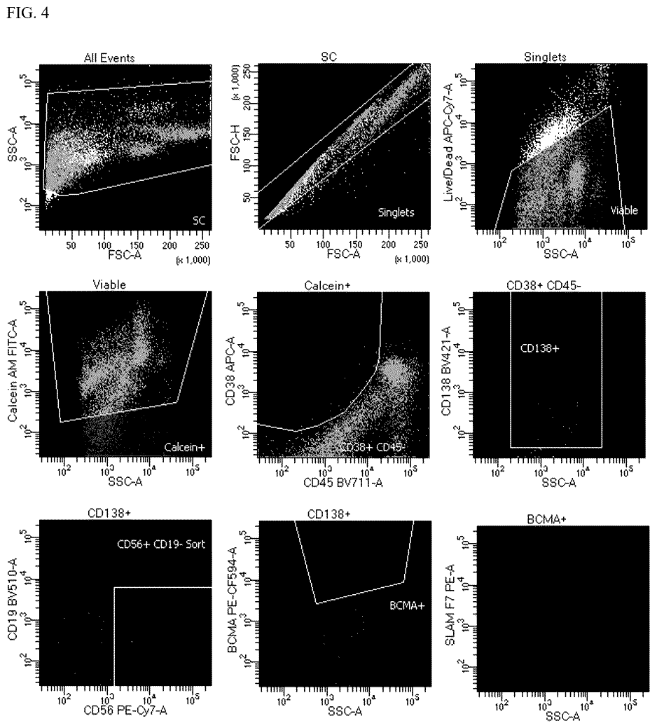

Samples were processed on a BD Symphony S6 UV Cell Sorter at the flow core of Dana-Farber Cancer Institute, and the tumor plasma cell fraction is sorted in PCR tubes (0.2 mL) or on 96-/384-well plates. The tumor PC population that was enriched is: CD38+ (CD38^{dim} if patient received anti-CD38 therapy), CD138+/dim (upon freezing), CD269+ (CD269^{dim} if patient received anti-BCMA therapy), CD19−, CD45−, CD56+/− (depending on previous clinical flow annotation), CD319+, LIVE/DEAD− and Calcein+. Examples of FACS gating strategies used for different samples are shown in FIGS. 2-4.

Sorting was performed on 3-8 patient samples, with a minimum expected of 5,000 events per sample. The fraction of interest was sorted into two mini pools of 25 CTCs or into one large bulk+mini-pools/single cells if the sample had a sufficient number of cells. A normal cell fraction was isolated for matched germline sequencing from total PBMCs or from the CD138-fraction. The number of cells collected from each sample is listed below in Table 6.

TABLE 6
Patient sample cell counts

| Patient | Pre-Treatment | Post-Treatment |
|---|---|---|
| No. | Cell No. | Cell No. |
| 1 | 50 | NA |
| 2 | 3165 | 20 |
| 3 | NA | NA |
| 4 | 591 | 7* |
| 5 | NA | NA |
| 6 | 25 | NA |
| 7 | 50 | NA |
| 8 | 151 | NA |
| 9 | 50 | NA |
| 10 | 50 | NA |
| 11 | 40 | NA |
| 12 | 40 | NA |
| 13 | 3627 | NA |
| 14 | 25 | NA |
| 15 | 5 | NA |
| 16 | 10263 | 3537 |

*The 7 cells obtained from Patient 4 post-treatment represent seven replicates of a single cell

For samples with less than 50 CTCs, Primary Template-Directed Whole Genome Amplification (PTWGA) was performed according to manufacturer's instructions using the ResolveDNA™ amplification kit or ResolveOME™ amplification kit (BioSkryb Genomics).

For samples that included more than 50 cells, a minipool of 50 cells was sampled, genomic DNA was extracted using the PicoPure DNA Extraction Kit (Arcturus).

In parallel, genomic DNA from matched germline samples from each patient were prepared for sequencing using the Monarch Genomic DNA Purification Kit (New England Biolabs).

DNA libraries were constructed using the NEBNext Ultra II FS DNA Library Prep Kit (New England Biolabs). initial library fragment sizes were assessed using the BioAnalyzer 2100 (Agilent Technologies), and yields were quantified by Qubit 3.0 fluorometer (Thermo Fisher Scientific) and qPCR (KAPA Library Quantification Kit).

Final sample libraries were normalized and pooled for matched tumor-normal whole-genome sequencing (WGS) to a target depth of 60× (bulk tumor), 30× (each mini pool and matched germline), and 10× (each single cell). WGS was performed on Illumina NovaSeq X instrument with loading on 10B or 25B flow cells, 2×150 bp paired-end reads.

After demultiplexing, reads were aligned to the GRCh38 reference genome with BWA MEM v0.7.15 (-K 100000000-p -v 3 -t 16 -Y parameters). After marking duplicates, base quality scores were recalibrated with GATK 4.2 BQSR without base quality quantization. WGS analysis was performed with the Cancer Genome Analysis workflow from the Cancer Program at Broad Institute of MIT and Harvard on an in-house cloud-based HPC system.

Mutations were detected with MuTect for single nucleotide variants (SNV), Strelka2 for small insertions and deletions (indels), and candidate mutations were then filtered for a series of known artifacts: Panel-of-Normal comprised of normal cells sequenced with the same protocol (n=40), Orientation Bias inducing artifacts (oxoG, FFPE), mapping artifacts (BLAT filter), enzymatic shearing cruciform variants. deTiN was used to estimate tumor-in-normal (TiN) contamination rate and rescue candidate mutations accordingly under 10% TiN.

Fingerprinting was used to confirm matching tumor-normal pairing. ConEst is used to detect exogenous DNA contamination. Candidate mutations were also matched against the bovine genome with BLAT to ensure samples were not contaminated from other species, in particular from FBS. Finally, candidate mutations were annotated with GATK Funcotator (v1.7).

Allelic copy number ratios were calculated with the HapASeg method (Hess, et al, Unpublished) modified to merge segments surrounding regions of allelic drop-outs (segments were removed from allelic SNP clustering if they contained less than 150 germline SNP). Briefly, HapASeg is a method that integrates copy-ratio segments and allelic imbalance obtained from the variant allele frequency of heterozygous SNPs from the matched normal. ABSOLUTE is used to estimate purity, ploidy, and cancer cell fraction of mutations and copy-number ratios, and all solutions from ABSOLUTE were manually reviewed before an experimenter selected the optimal one based on the clonality of copy-number alterations and point mutations.

When several samples were available for the same participant, subclonal structures were deciphered with the PhylogicNDT suite of tools. When several cells were sequenced from the same samples, the Sequoia method was used to build a phylogenetic tree.

Structural variants (SVs) were detected with Manta, SvABA, and dRanger/BreakPointer. Consensus lists of candidate SVs were then established as previously described with the exception of the 10% VAF filter, which was set to 0% to increase sensitivity to detect immunoglobulin rearrangements. In particular, these were filtered based on genomic locations found in Supplementary Table S1. Finally, all candidate SVs from the deep WGS cohorts were manually reviewed with IGV.

Clinical characteristics of each patient were assessed at baseline, and responses to therapy were determined according to the IMWG Uniform Response Criteria for MM.

We confirmed that CTC samples originated from the tumor in all 16 cases (median tumor purity: 99%, range 22% to 100%) and B cell receptor in most cases (n=14/16, 88%). Initiating events such as translocation and hyperdiploidy were detected in the majority of all cases (n=13/16, 81%). Twelve patients had a Fluorescence In Situ Hybridization (FISH) test performed earlier during their disease course. We could confirm all of their cytogenic findings and discover additional high-risk abnormalities at the RRMM time point (del(17p), n=1; gain(1q), n=1). Because WGS is unbiased, we also detected point mutations at expected rates known in MM drivers in 13 out of 16 tumors (81%) showing the reliability of this assay (NRAS/KRAS [6/16; 40%], TP53 [3/16; 19%]; DIS3 [3/16; 19%]; SP140 [1/16; 6%], etc.). Of note none of these patients had a detectable mutation in GPRC5D or TNFRSF17 (which encode BCMA), even in those who had already received an anti-BCMA treatment (ide-cel CAR-T, n=4). Lack of target mutation was consistent with the depth of response achieved (CR, n=10; VGPR, n=1; PR, n=2; SD, n=2 [anti-GPRC5D TCE]; non-relapsed death, n=1) including in one patient switching from an anti-BCMA CAR-T to an anti-BCMA TCE (VGPR). The absence of mutations affecting BCMA after CAR-T suggests that the previous lines of therapy did not select for a mutated clone that expanded during therapy.

We then investigated whether resistant clones were present prior to disease relapse, which could inform decisions regarding early therapy changes period to that aim we longitudinally characterized CTCs in three patients undergoing treatment with immunotherapies. The first patient receiving anti BC MATC only achieved SSD. Despite a 65% decrease in CTC counts, we already detected the selection of a clone bearing a TP53 nonsense mutation R34 two star and a complex karyotype before progression. In another patient receiving anti-BCMA RT who achieved a sCR (MRD positive at 10-6), we could perform PTWGA and WGS on 7 single-cells. Of those, 5 were confirmed to originate from the tumor clone, but showed no mutation in TNFRSF17. We also sequenced MRD cells after six rounds of therapy (CR) and did not find mutations in TNFRSF17, suggesting another mechanism of treatment escape at this stage.

### Example 2—Identification of and Correction for False Positive Detection at Pseudo-Palindromic Genomic Regions

Within sequencing results, it was identified that regions including DNA palindromes had a higher-than-expected rate of detection of mutation. DNA palindromes are DNA sequences that are reverse complements of themselves (e.g., ACGT but not AAAA or ACCA). Enzymatic methods of preparing sequencing libraries are known to introduce false positive mutations at pseudo-palindromic regions of DNA. Pseudo-palindromic regions additionally show a characteristic referred to as “soft clipping” in sequencing results, which allows for detection and elimination of pseudo-palindromic regions when desired. Here, an additional analysis pipeline was applied to CTC WGS samples to identify and remove false positive mutations accumulated in pseudo-palindromic regions.

In single cells sequencing of male cells, chromosomes X and Y include only clonal mutations because only one copy of each of the X and Y chromosomes are found within each cell. In other words, each of the X and Y chromosomes from a single cell should only have exactly one genomic sequence, unlike other chromosomes that may include different alleles within the same gene.

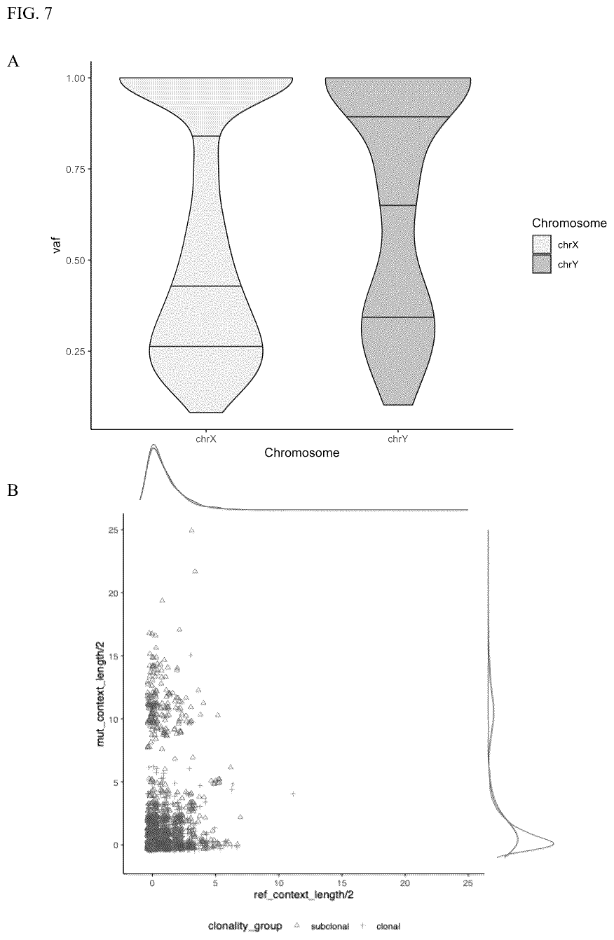

To identify and extract potential false positive mutations in pseudo-palindromic regions, data from 23 single male cells were analyzed to identify subclonal mutations. Subclonal mutations were identified as mutations having a vaf of less than approximately 0.75 (FIG. 7A). Mutations that had a vaf of greater than approximately 0.75 were considered to be clonal. Subclonal and clonal mutation locations were mapped against the longest palindrome of the germline and the longest palindrome of the tumor (FIG. 7B). From this analysis, it was identified that approximately 10% of all mutations identified using the enzymatic sequencing library preparation method described in Example 1 could be attributed to pseudo-palindromic false positive mutations.

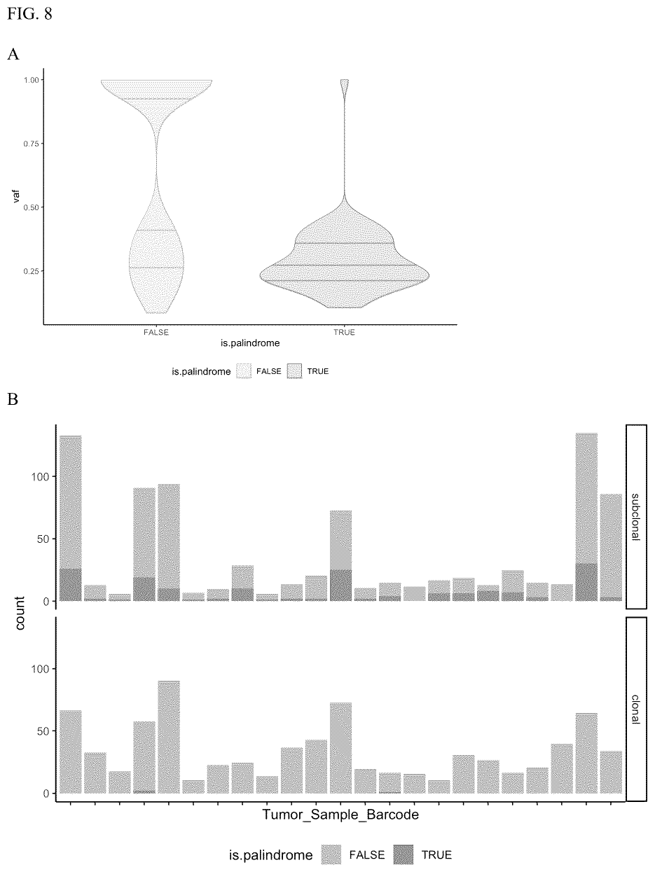

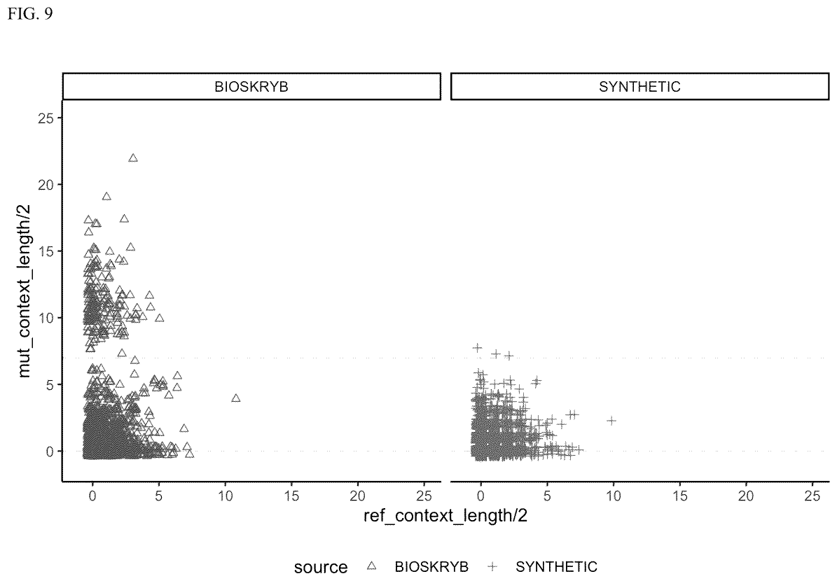

Next, the correlation between palindrome mutations and subclonal mutations was analyzed. As is shown in FIG. 8, nearly all subclonal mutations were mapped to palindromic regions. In contrast, naturally occurring mutations rarely occur within palindromic regions (FIG. 9). This suggested that palindromes, and specifically pseudo-palindromic mutations introduced by the BioSkryb sequencing library preparation, were likely the source of subclonal mutations.

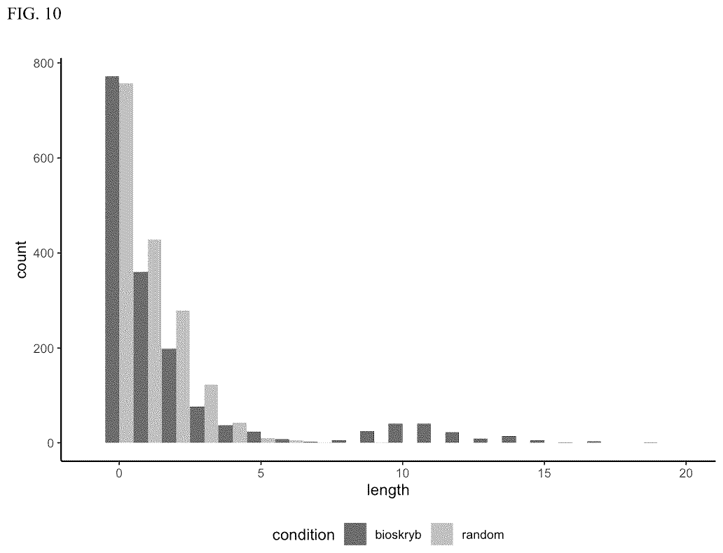

To help identify pseudo-palindromic mutations introduced by the BioSkryb sequencing library preparation, the length of each identified clonal (labeled as random) and subclonal (labeled as bioskryb) palindrome was mapped (FIG. 10). Notably, the sequencing library preparation introduced longer palindromes than were commonly found naturally, with some palindromic sequencing having a length of 9-15 nucleotides. This indicates that longer palindromic regions may be more suspect than short palindromic regions. It was suspected that the sequencing library preparation kit introduced longer palindromes by resolving quasi-palindromic regions into perfect pseudo-palindromic regions by introducing one or more mutations.

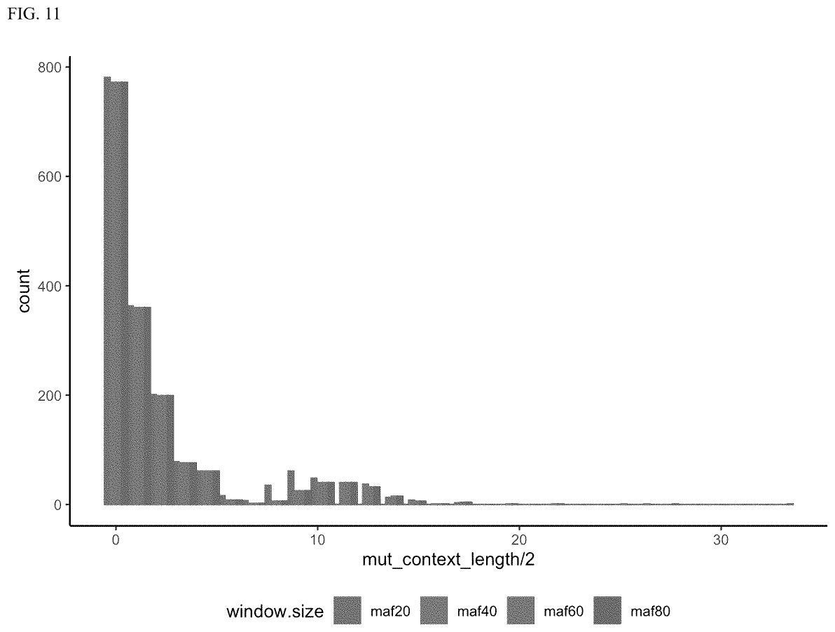

To filter out the false positive mutations introduced by this pseudo-palindromic mechanism, reading windows of 20 bp, 40 bp, 60 bp, and 80 bp were tested (FIG. 11). It was found that a reading window of 40 bp identified the majority of palindromes at an acceptable computational expense. Using this information, an updated analysis pipeline was prepared to identify and filter palindromes in DNA prepared using the BioSkryb sequencing library preparation kit. In brief, the algorithm take the following steps. First, the genomic coordinates of mutations are read. The germline neighbor sequence at each mutation is extracted from the known human genome sequence, and the mutation is introduced to the reference sequence. Next, the longest palindrome around the mutation is identified. This identification is repeated on 1,000 permutations of the DNA context. If the permutation P-value is found to be greater than 0.0.5, filtering is implemented. To filter the mutation, the palindrome is decoded with more precision. Quality control data on the palindrome is collected, and mutations that are identified as turning quasi-palindromes into full palindromes are filtered out.

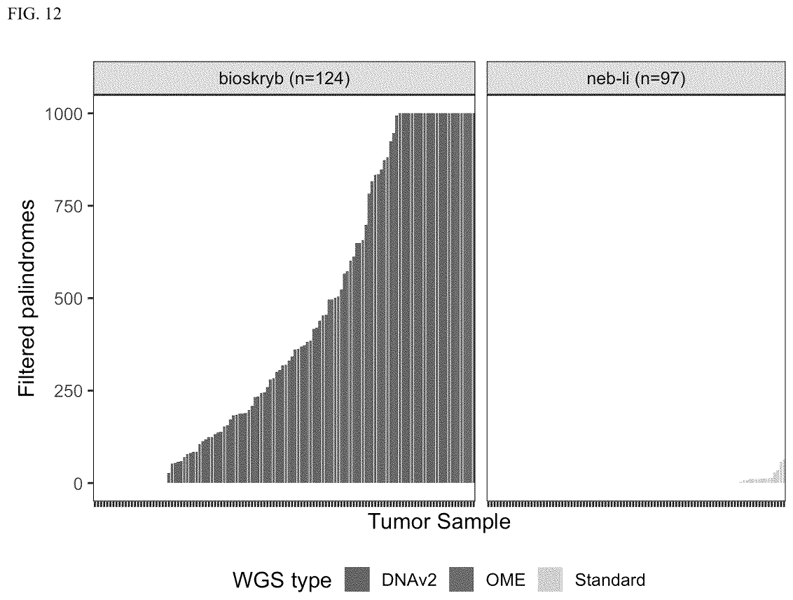

To further confirm whether the source of these pseudo-palindromic mutations was the BioSkryb sequencing preparation kit, filtered palindromes were identified samples prepared either using the BioSkryb kit or the New England Biolabs kit. While some palindromes were identified in the sample prepared using a standard kit, many more were identified in the samples prepared using the BioSkryb kit (FIG. 12). This further supported the idea that the BioSkryb kit was a source of the pseudo-palindromic false positive mutations.

### Example 3—Identification of “Persister” Cells and Evolution of Tumor Clones During Immunotherapy and at Minimal Residual Disease Timepoints Decoded by Single-Cell Whole-Genome Sequencing in Multiple Myeloma

Tumor plasma cells from 11 patients with high-risk smoldering MM (HR-SMM, n=9) or relapsed/refractory MM (RRMM, n=2) were isolated using 9-color fluorescence-activated cell sorting (FACS) after magnetic bead enrichment for CD138 on BM and PB samples. For scWGS, ResolveDNA (BioSkryb Genomics) was used to amplify DNA prior to library preparation. All DNA libraries were prepared using Ultra II FS (New England Biolabs), and sequencing was performed at a depth of 60× coverage (WGS) or 10× coverage per single cell (scWGS). Subclonal dynamics were analyzed using the PhylogicNDT suite, and phylogenetic trees were inferred with the Sequoia method.

Longitudinal WGS at baseline, MRD timepoints, and/or relapse in patients receiving DRVd (n=6), Teclistamab (n=4), or Cilta-cel (n=1) was performed. In patients treated with Daratumumab, CD38 expression was low in tumor samples collected during treatment (n=3) but high in those collected after the end of treatment (n=3). In one of the 3 on-treatment samples, a heterozygous CD38 gene deletion was identified in 3 out of 3 single tumor cells, from 2 independent clonal branches. No genomic alterations at the CD38 locus were detected in the remaining samples, suggesting that they could be rechallenged with CD38-targeted therapies irrespective of clinical response and clonal selection. Notably, the CD38 deletion was detected at an MRD timepoint and was later confirmed by bulk sequencing at disease progression. In contrast, scWGS of another hyperdiploid patient who remained MRD+ revealed unexpected subclonal chromosomal heterogeneity (including within trisomies 9 and 15) across all 22 single cells analyzed at the baseline timepoint-findings that were not detectable by bulk sequencing. After 12 cycles of therapy, the patient achieved a very good partial response (VGPR), and we profiled 12 tumor cells as part of the planned MRD assessment, which were CD38 low/negative. The tumor cells branched out of the clone found at baseline and harbored additional copy-number abnormalities (del(16q) and del(6q)).

Across the five patients treated with Teclistamab (n=4) or Cilta-cel (n=1), BCMA expression remained high throughout treatment despite four of them not responding or relapsing after treatment. The fifth patient received Teclistamab, we profiled 4 single cells collected after 6 months of therapy (MRD negative 10-5, positive 10-6). We identified a new heterozygous loss of chromosome 16 (encompassing BCMA) in the single BCMA+tumor plasma cell, while 3 other candidate cells were found to be normal plasma cells (false positives by FACS). The patient continued therapy and achieved CR and MRD negativity (<10-6), suggesting that even cells with a single copy of BCMA remaining can still be eliminated post-therapy.

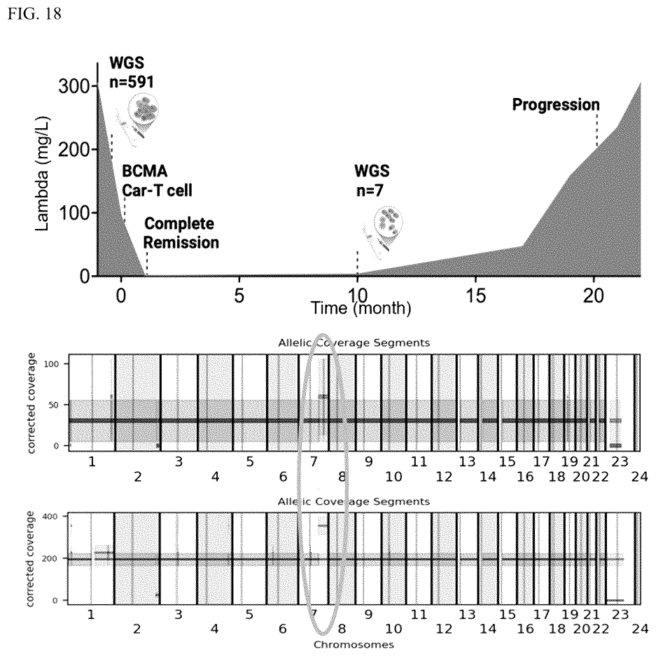

Lastly, a patient receiving Cilta-cel achieved CR with MRD positivity (>10-6). We performed WGS of 7 and 30 BCMA+single circulating tumor cells at 10-months and 18-months post-infusion, respectively, prior to clinical progression. Our analysis revealed genomically mature, persistent tumor cells lacking BCMA mutations, branching from the baseline tumor and acquiring additional mutations. Relapsed disease at 18 months originated from several of the clones already present at 10 months (FIG. 18).

Using scWGS, we profiled persistent tumor cells during immunotherapy with minimal disease burden available. Subclonal analysis suggested that these “persister” cells primarily branched from a genomically mature, late clone-some acquiring additional mutations targeting the antigen as early as 6 months into therapy-rather than from an ancestral population.

Those skilled in the art will recognize, or be able to ascertain, using no more than routine experimentation, numerous equivalents to the specific substances and procedures described herein. Such equivalents are considered to be within the scope of this disclosure. The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the teachings of the invention without departing from the essential scope thereof. One skilled in the art will readily recognize from such discussion, and from the accompanying drawings and claims, that various changes, modifications and variations can be made therein without departing from the spirit and scope of the present teachings as defined in the following claims.

1.-20. (canceled)

21. A method for monitoring minimum residual disease (MRD) in a subject, comprising:
(a) isolating one or more cells from a biological sample of said subject using fluorescence-activated cell sorting (FACS) to provide a cell sample;
(b) performing whole genome amplification on said cell sample to provide an amplified genome;
(c) performing whole genome sequencing on said amplified genome thereby producing one or more sequencing reads; and
(d) monitoring MRD in said subject, based at least in part on detecting one or more gene abnormalities associated with MM or a precursor thereof in said one or more sequencing reads.

22. The method of claim 21, comprising selecting or administering a treatment based on MRD of said subject determined from said monitoring MRD in said subject.

23. The method of claim 22, wherein said treatment comprises surgery, a chemotherapeutic agent, radiation, bone marrow transplant, an immunotherapy, a hematopoietic stem cell transplant, an antibody, or any combination thereof.

24. The method of claim 21, wherein said one or more gene abnormalities comprise a translocation, a copy number variation (CNV), a single nucleotide variation (SNV), a insertion or deletion (indel), or any combination thereof.

25. The method of claim 21, wherein said one or more cells are isolated by labeling said one or more cells with one or more fluorescently labeled antibodies having affinity to one or more of CD138, CD38, CD56, CD319, CD19, CD45, or CD269.

26. The method of claim 21, wherein said biological sample comprises peripheral blood or a bone marrow aspirate.

27. The method of claim 21, wherein said one or more cells comprise circulating tumor cells isolated from peripheral blood of said subject, or tumor plasma cells isolated from a bone marrow aspirate of said subject.

28. The method of claim 21, wherein said whole genome amplification comprises primary template-directed whole genome amplification (PTWGA), and wherein said cell sample comprises about 25 cells or fewer.

29. The method of claim 21, wherein said subject has been previously treated with a chimeric antigen receptor T cell (CAR-T) or a T cell engager (TCE) directed against BCMA, GPRC5D, CD38, or an anti-CD38 monoclonal antibody or any combination thereof, and wherein said isolating comprises selecting cells that are CD269dim, CD38dim, or both, based on said previous treatment.

30. A method for identifying a resistance to a treatment of a subject with minimum residual disease (MRD), comprising:
(a) isolating one or more cells from a biological sample of said subject using fluorescence-activated cell sorting (FACS) to provide a cell sample;
(b) performing amplification on a nucleic acid molecule of said cell sample to provide a plurality of nucleic acid molecules; and
(c) identifying said resistance to said treatment of said subject with MRD, based at least in part on detecting one or more gene abnormalities associated with multiple myeloma or a precursor thereof in said plurality of nucleic acid molecules.

31. The method of claim 30, comprising performing whole genome sequencing on said plurality of nucleic acid molecules.

32. The method of claim 30, comprising selecting or administering a treatment based on MRD of said subject determined from said monitoring MRD in said subject.

33. The method of claim 32, wherein said treatment comprises surgery, a chemotherapeutic agent, radiation, bone marrow transplant, a hematopoietic stem cell transplant, an antibody, or any combination thereof.

34. The method of claim 30, wherein said subject has been previously treated with a chimeric antigen receptor T cell (CAR-T) or a T cell engager (TCE) directed against BCMA, GPRC5D, CD38, or an anti-CD38 monoclonal antibody or any combination thereof, and wherein said isolating comprises selecting cells that are CD269dim, CD38dim, or both, based on said previous treatment.

35. The method of claim 30, wherein said biological sample is obtained from said subject at a plurality of time points before, during, after said treatment, or any combination thereof, and wherein said method further comprises monitoring tumor clonal evolution based on said one or more gene abnormalities detected at each of said plurality of time points.

36. The method of claim 30, wherein said one or more gene abnormalities associated with said resistance comprise a mutation in BCMA (TNFRSF17), GPRC5D, CD38, TP53, or any combination thereof, or a deletion of a chromosomal region encoding any one or more of said foregoing mutations.

37. The method of claim 30, wherein said amplification comprises primary template-directed whole genome amplification (PTWGA), and wherein said cell sample comprises about 25 cells or fewer.

38. The method of claim 37, wherein said PTWGA uses exonuclease-resistant terminators to produce double-stranded amplification products, thereby limiting amplification bias, allelic skewing, propagation of replication errors, or any combination thereof.

39. The method of claim 30, wherein said one or more cells comprise circulating tumor cells isolated from peripheral blood of said subject, or tumor plasma cells isolated from a bone marrow aspirate of said subject.

40. The method of claim 30, wherein said subject has been previously treated with a chimeric antigen receptor T cell (CAR-T) or a T cell engager (TCE) directed against BCMA, GPRC5D, CD38, or an anti-CD38 monoclonal antibody, or any combination thereof, and wherein said isolating comprises selecting cells that are CD269dim, CD38dim, or both, based on said previous treatment.
